(19) United States
(12) Patent Application Publication
(10) Pub. No.: US 2009/0099468 A1
(43) Pub. Date: Apr. 16, 2009

(54) Automated Processing of Electrophysiological Data

(76) Inventors: Aravinda Thiagalingam, New South Wales (AU); Pramesh Kovoor, New South Wales (AU); David Leslie Ross, New South Wales (AU)

Correspondence Address: Christine A. Lekutis;Medlen & Carroll LLP, 101 Howard Street, Suite 350, San Francisco, CA, 94105, US

(21) Appl. No.: 11/793,573

(22) PCT Filed: Dec. 20, 2005
(86) PCT No.: PCT/AU05/01925

§ 371 Date: May 6, 2008

(30) Foreign Application Priority Data
Dec. 21, 2004 (AU) 2004907260

(51) Int. Cl. A61B 5/0402

(52) U.S. Cl. 600/515

(57) ABSTRACT

A method (100), an apparatus, and a computer program product are disclosed for automated processing of intracardiac electrophysiological data. The method (100) comprises the steps of: recording (112) electrogram data and corresponding spatial location data of an electrode recording the electrogram data, the recorded electrogram data comprising a plurality of beats; defining (110) at least one reference channel containing a reference beat (114) for determining temporal locations and against which beats of the recorded electrogram data are compared; examining the recorded electrogram data and defining a temporal location for each beat of the recorded electrogram data; creating an index (116) of the temporal locations and other information of the beats within the recorded electrogram data; analysing (118) in real-time at least one electrophysiological feature of the recorded electrogram data suggestive of a physiological condition; and providing an updated index wherein the other information comprises results of the analysis.

### TECHNICAL FIELD

The present invention relates generally to systems for obtaining electrophysiological data and more particularly to systems for processing electrocardiac signals.

### BACKGROUND

Cardiac electrophysiological procedures have become increasingly complex as clinicians treat challenging conditions such as atrial fibrillation and ventricular tachycardia. Atrial fibrillation is a form of heart arrhythmia involving the loss of synchrony between the atria and ventricles caused by re-entrant circuits. Ventricular tachycardia is a form of rapid heart beat involving any rapid rhythm faster than 100-120 beats per minute arising in the ventricle. In many of these cases, the abnormal myocardium that requires treatment is in close proximity to a region of a scar. To treat these disorders, several electrophysiology systems have been proposed that seek to determine the location of a catheter tip within the heart chamber of interest during diagnosis and/or treatment. These systems can be used to create a 3-dimensional map showing the location of a scar through a process of sequentially collecting data from different regions. However, this methodology is slow and labour intensive requiring approximately 150-200 sequentially acquired points (see Marchlinsky, Zado, Dixit and others, “Electroanatomic Substrate and Outcome of Catheter Ablative Therapy for Ventricular Tachycardia in Setting of Right Ventricular Cardiomyopathy”, Circulation 2004, Vol. 110, pp. 2293-2298).

A small number of systems have been proposed to automate cardiac electrogram analysis, e.g. U.S. Patent Application Publication No. 2004/0122332 A1. U.S. Patent Application Publication No. 2004/0122332 A1 filed on 2 Nov. 2001 in the name of Macadam et al and published on 24 Jun. 2004 describes a system to track the position of ectopic atrial beats (i.e., premature atrial beats) and also perform pace mapping. The system compares paced or ectopic beats with a known reference beat using Pearson's correlation to determine how likely the ectopic or paced activity is to have originated from the same location as the reference beat. Disadvantageously, the system is restricted in the situations that the system that can be used (i.e. pace mapping and analysis of the surface ECG). The system is designed to assist the operator to perform sequential mapping (i.e. the electrograms are analysed at a given location before moving to the next location) and hence suffers from the same delays that manual systems experience.

A number of systems have been proposed that attempt to automate the process of pace mapping. Pace mapping is performed during a cardiac electrophysiology procedure to locate the site of origin of a given arrhythmia. The operator moves the catheter and paces from various locations within the heart. The ECG signals recorded during these pacing episodes are compared to the ECG signals recorded during the arrhythmia. If the pacing catheter is near the origin of the arrhythmia, the two sets of ECG signals should be the same or quite similar. This is a time consuming process as there may be twelve ECG signals to compare with each other at each pacing site.

A number of systems seek to automatically correlate the two sets of ECG signals including: U.S. Patent Application Publication Nos. 20020065459 A1 and 20040127805 A1 published respectively on 30 May 2002 and 1 Jul. 2004 in the name of Macadem et al; U.S. Pat. No. 6,690,963 issued on 10 Feb. 2004 in the name of Ben-Haim et al; and U.S. Patent Application Publication No. 20040059237 published on 25 Mar. 2004 to Narayan et al. The systems automatically correlate two sets of ECG signals. These systems are designed to compare a small number of the electrograms that occur during a limited time frame (during one episode of pacing) with a single beat occurring during the arrhythmia.

U.S. Pat. No. 6,690,963 also describes the creation of a variety of spatial maps using an electromagnetic catheter location system. However the data used to create these maps must be acquired in a sequential fashion with user intervention required to select suitable beats and suitable locations.

U.S. Patent Application Publication Nos. 20040122332 and 20040127805 A1 also describe a system for data management that allows the operator to organise information collected from different locations within the heart and display spatial maps with selected subsets of this data. This system may allow the operator to view electrogram data before and after an intervention, but these data points have to be acquired individually by the user.

U.S. Patent Application Publication No. 20040059237 also describes a methodology to detect entrainment with concealed fusion (ECF), which is a modification of the previously described pace mapping technique.

U.S. Pat. No. 6,751,492 issued on 15 Jun. 2004 to Ben-Haim describes methods to construct cardiac maps showing the spatial distribution of a variety of physiological variables, such as myocardial thickness, local wall tension or chemical concentrations. One of the intended uses of these methods is to determine the optimal site to pace the heart. The correlation coefficient is used to differentiate different beats for situations where there are frequent ectopic beats occurring during the mapping procedure. These methods again rely on sequentially acquired data that has been selected by the clinician performing the procedure.

U.S. Pat. No. 6,285,898 issued on Sep. 4, 2001 to Ben-Haim describes a method of computer-aided diagnosis using a stored library of cardiac maps and the associated diagnosis. The map that is acquired for a particular patient can be compared with stored maps to determine which stored map the acquired map most closely correlates with. This method is also likely to be of more use to less experienced clinicians as highly skilled operators often have their own stored library of maps in memory and hence do not require such an aid. The method may aid with the diagnosis of the patient in some cases, but the method still requires the initial patient map to be constructed using a sequential and time consuming methodology.

Thus, a need clearly exists for a system that enables an operator to fully utilise available data, rather than just selected beats, and that can compile and analyse this data into an easily accessible format, allowing rapid and accurate mapping of the cardiac chambers.

### SUMMARY

In accordance with a first aspect of the invention, there is provided a method of automated processing of intracardiac electrophysiological data. The method comprises the steps of: recording electrogram data and corresponding spatial location data of an electrode recording the electrogram data, the recorded electrogram data comprising a plurality of beats; defining at least one reference channel containing a reference beat against which beats of the recorded electrogram data are compared; examining the recorded electrogram data and defining a temporal location for each beat of the recorded electrogram data; creating an index of the temporal locations and other information of the beats within the recorded electrogram data; analysing in real-time at least one electrophysiological feature of the recorded electrogram data suggestive of a physiological condition; and providing an updated index wherein the other information comprises results of the analysis.

The physiological condition may comprise a local abnormality. The local abnormality may comprise myocardial scarring.

The method may further comprise the step of generating a scatter plot graphically representing information based on the recorded electrogram data.

The method may further comprise the step of generating dependent upon the updated index a multidimensional substrate map comprising a plurality of vertices each having a corresponding spatial location and graphically representing information based on the recorded electrogram data. At least a portion of the graphically represented information may represent scarred myocardial tissue. At least a portion of the graphically represented information may represent healthy myocardial tissue. The information may be graphically represented using colour. The multidimensional substrate map is a 3-dimensional substrate map.

The method may further comprise the step of excluding from the substrate map data for beats recorded within the cardiac chamber that have insufficient contact with a wall of the cardiac chamber.

1. The excluding step comprises using a convex hull algorithm to select beats recorded at extreme locations for display in the substrate map. The method may further comprise the step of triggering an alarm dependent upon the updated index.

The method may further comprise the step of choosing at least one electrogram channel to be recorded, dependent upon the application. The chosen channel may comprise at least one of ECG channels, a ventricular reference channel, an atrial reference channel, and a distal mapping catheter.

The method may further comprise the step of analysing as a background task at least one electrophysiological feature of the recorded electrogram data.

The real-time analysing step may comprise checking the recorded electrogram data for at least one feature suggestive of a fault condition or an artefact.

The defining step may comprise determining fiducial points that define the temporal location of each beat recorded within the reference channel. The method may further comprise the steps of calculating a differentiated reference electrogram and finding points at which the differentiated reference electrogram exceeds a predefined beat threshold to determine the fiducial points.

The step of calculating the differentiated electrogram may comprise subtracting a data point from the recorded electrogram data at a specified temporal location before the time zero point from a data point from the recorded electrogram data at another specified temporal location after the time zero point. The step of calculating the differentiated electrogram may further comprise dividing the subtraction value by the number of data samples between the data points and multiplying the result by a sampling rate of the recorded electrogram data.

The real-time analysing step may comprise determining an analysis segment of the recorded electrogram data. The analysis segment is created as a sub-segment a predetermined time before and after the fiducial point for each beat in the recorded electrogram data. The real-time analysing step may comprise finding a minimum slope once for each analysis segment for each beat. The real-time analysing step may comprise detecting when a local activation occurs in the analysis segment in reference to the fiducial point in the beat of the recorded electrogram data. The detecting step may comprise determining a temporal location at which the minimum slope occurs.

The real-time analysing step may comprise correlating at least one reference beat of the reference channel with beats of the recorded electrogram data.

The feature analysed in real time may comprise the minimum slope in a differentiated unipolar electrogram in a distal mapping catheter electrode.

In accordance with a further aspect of the invention, there is provided a computer program product comprising a computer readable medium having recorded therein a computer program for automated processing of intracardiac electrophysiological data. The computer program product comprises: a computer program code module for recording electrogram data and corresponding spatial location data of an electrode recording the electrogram data, the recorded electrogram data comprising a plurality of beats; a computer program code module for defining at least one reference channel containing a reference beat against which beats of the recorded electrogram data are compared; a computer program code module for examining the recorded electrogram data and defining a temporal location for each beat of the recorded electrogram data; a computer program code module for creating an index of the temporal locations and other information of the beats within the recorded electrogram data; a computer program code module for analysing in real-time at least one electrophysiological feature of the recorded electrogram data suggestive of a physiological condition; and a computer program code module for providing an updated index wherein the other information comprises results of the analysis.

A computer program product may be provided in accordance with other aspects of the foregoing method.

In accordance with another aspect of the invention, there is provided an apparatus for automated processing of intracardiac electrophysiological data. The apparatus comprises: a module for recording electrogram data and corresponding spatial location data of an electrode recording the electrogram data, the recorded electrogram data comprising a plurality of beats; a module for defining at least one reference channel containing a reference beat against which beats of the recorded electrogram data are compared; a module for examining the recorded electrogram data and defining a temporal location for each beat of the recorded electrogram data; a module for creating an index of the temporal locations and other information of the beats within the recorded electrogram data; a module for analysing in real-time at least one electrophysiological feature of the recorded electrogram data suggestive of a physiological condition; and a module for providing an updated index wherein the other information comprises results of the analysis.

An apparatus may be provided in accordance with other aspects of the foregoing method.

In accordance with still another aspect of the invention, there is provided a system for automated processing of intracardiac electrophysiological data. The system comprises: a memory for storing data and a computer program; and a processor coupled to the memory executing the computer program. The computer program comprises instructions for: recording electrogram data and corresponding spatial location data of an electrode recording the electrogram data, the recorded electrogram data comprising a plurality of beats; defining at least one reference channel containing a reference beat against which beats of the recorded electrogram data are compared; examining the recorded electrogram data and defining a temporal location for each beat of the recorded electrogram data; creating an index of the temporal locations and other information of the beats within the recorded electrogram data; analysing in real-time at least one electrophysiological feature of the recorded electrogram data suggestive of a physiological condition; and providing an updated index wherein said other information comprises results of the analysis.

The system may further comprise a monitor for displaying graphical data. The computer program may further comprise instructions for generating dependent upon the updated index a multidimensional substrate map comprising a plurality of vertices each having a corresponding spatial location and graphically representing information based on the recorded electrogram data.

The computer program may further comprise instructions for triggering an alarm dependent upon the updated index. The system may further comprise an alarm mechanism producing an audible, visual, or both alarm signal.

### BRIEF DESCRIPTION OF THE DRAWINGS

Embodiments of the invention are described hereinafter with reference to the drawings, in which:

FIG. 1 is a high-level flow diagram illustrating the operations performed in automated substrate mapping, which may be implemented in a computer program;

FIG. 2A is a plot illustrating an electrogram review window containing a reference channel and an intracardiac channel having four beats;

FIG. 2B is a plot illustrating a substrate map created using the electrograms of FIG. 2A;

FIG. 2C is a graphical depiction of a heart with a mapping catheter disposed within the left ventricle and the corresponding recording locations (1, 2, 3, 4) for the beats of FIG. 2A;

FIG. 3 is a screenshot illustrating an electrogram review window;

FIG. 4 is a plot illustrating an automated substrate map window;

FIG. 5 contains a plot of an automated 3-dimensional substrate map and an image of a sheep's heart from which the map was generated;

FIG. 6 illustrates electrogram data, analysed beat data, and geometry (map) data;

FIG. 7 is a flow diagram illustrating the process of data recording;

FIG. 8 is a plot diagrammatically depicting in black and white the substrate map of FIG. 5;

FIG. 9A is a flow diagram illustrating the process of data synthesising;

FIG. 9B is a plot showing an original signal and the resulting differentiated signal produced by the process of FIG. 9A;

FIG. 10 contains plots illustrating the process of beat detection;

FIG. 11A is a flow diagram illustrating the process of beat detection;

FIG. 11B is a flow diagram illustrating in detail the automatic reference beat selection algorithm of step 1116 in FIG. 11A;

FIG. 12A is a flow diagram illustrating the process of beat classification of step 1162 in FIG. 11B;

FIG. 12B is a plot of a reference beat having a fiducial point depicted between the reference, start and end times;

FIG. 12C shows an analysis (test) electrogram offset from a reference electrogram;

FIG. 13 is a flow diagram providing an overview of an electrogram analysis process;

FIG. 14A is a flow diagram illustrating the electrogram fault analysis process of FIG. 13;

FIG. 14B contains plots illustrating electrograms undergoing testing for saturation and voltage spikes;

FIG. 15A is a flow diagram illustrating the main electrogram analysis process of FIG. 13;

FIG. 15B contains plots illustrating electrograms and analysis results;

FIG. 16 is a flow diagram illustrating the extended analysis process of FIG. 13;

FIG. 17A is a flow diagram illustrating the multi-chamber analysis process of FIG. 13;

FIG. 17B is a plot showing a reference channel and an atrial intracardiac channel;

FIG. 18 is a flow diagram illustrating an activation analysis process of FIG. 13;

FIG. 19 is a flow diagram illustrating an overview of a data display process;

FIG. 20 illustrates the process of geometry framework construction of step 1910 in FIG. 19;

FIG. 21 is a flow diagram illustrating a colour assignment process of step 1912 in FIG. 19;

FIG. 22 is a flow diagram illustrating a substrate map display process of FIG. 19 step 1914;

FIG. 23A is a flow diagram illustrating a substrate map review process;

FIG. 23B is a plot illustrating a region of interest in a substrate map and the associated zoomed electrogram map; and

FIG. 24 is a block diagram of a general-purpose computer system with which embodiments of the invention may be practiced.

### DETAILED DESCRIPTION

Methods, apparatuses, and computer program products are disclosed for automated processing of electrophysiological data. In the following description, numerous specific details, including particular spatial coordinate systems, colours, graphical representation mechanisms, and the like are set forth. However, from this disclosure, it will be apparent to those skilled in the art that modifications' and/or substitutions may be made without departing from the scope and spirit of the invention. In other circumstances, specific details may be omitted so as not to obscure the invention.

Where reference is made in any one or more of the accompanying drawings to steps and/or features, which have the same reference numerals, those steps and/or features have for the purposes of this description the same function(s) or operation(s), unless the contrary intention appears. In the context of this specification, the word “comprising” has an open-ended, non-exclusive meaning: “including principally, but not necessarily solely”, but neither “consisting essentially or” nor “consisting only of”. Variations of the word “comprising”, such as “comprise” and “comprises”, have corresponding meanings.

The term “cardiac chamber” is used hereinafter to refer to the anatomical area analysed with the embodiments of the invention in the interest of brevity. The utility of this invention, however, is not limited to the cardiac chambers alone (eg. this software can be used to investigate the veins and arteries associated with the heart such as the pulmonary veins).

The method for automated mapping and indexing of electrophysiological data may be implemented in modules. A module, and in particular its functionality, can be implemented in either hardware or software. In the software sense, a module is a process, program, or portion thereof that usually performs a particular function or related functions. Such software may be implemented in C, C++, JAVA, ADA, Fortran, for example, but may be implemented in any of a number of other programming languages/systems, or combinations thereof. The software may be implemented to take advantage of expert system technology, in which a set of rules for identifying the location of critical regions within the heart is stored in a database. This set of rules can therefore be viewed and modified by the user allowing advanced users to enhance the software using their own knowledge and clinical experience. The software may also incorporate neural networks to aid in data processing.

In the hardware sense, a module is a functional hardware unit designed for use with other components or modules. For example, a module may be implemented using discrete electronic components, or it can form a portion of an entire electronic circuit such as a Field Programmable Gate Arrays (FPGA), Application Specific Integrated Circuit (ASIC), and the like. A physical implementation may also comprise configuration data for a FPGA, or a layout for an ASIC, for example. Still further, the description of a physical implementation may be in EDIF netlisting language, structural VHDL, structural Verilog or the like. Numerous other possibilities exist. Those skilled in the art will appreciate that the system can also be implemented as a combination of hardware and software modules.

Some portions of the description are explicitly or implicitly presented in terms of algorithms and representations of operations on data within a computer system or other device capable of performing computations, e.g. a personal digital assistant (PDA), and the like. These algorithmic descriptions and representations may be used by those skilled in the data processing arts to convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical, magnetic, or electromagnetic signals capable of being stored, transferred, combined, compared, and otherwise manipulated.

Principally for reasons of common usage, it has proven convenient at times to refer to these signals as bits, values, elements, symbols, characters, terms, numbers, or the like. However, the above and similar terms are to be associated with the appropriate physical quantities and are merely convenient labels applied to these quantities. Unless specifically stated otherwise, and as apparent from the following, it will be appreciated that throughout the present specification, discussions utilizing terms such as “executing”, “loading”, “defining”, “recording”, “creating”, “storing” “analysing”, “displaying”, “mapping”, “constructing”, “advancing”, “moving” or the like, refer to the actions and processes of a computer system, or similar electronic device. The computer system, or similar electronic device, can manipulate and transform data represented as physical (electronic) quantities within the registers and memories of the computer system into other data similarly represented as physical quantities within the computer system memories, registers, or other information storage, transmission or display devices.

The present specification also discloses a system or an apparatus for performing the operations of these algorithms. Such a system may be specially constructed for the required purposes, or may comprise a general-purpose computer or other similar device selectively activated or reconfigured by a computer program stored within its memory. The algorithms presented herein are not inherently related to any particular computer or other apparatus. Various general-purpose machines may be used with programs in accordance with the teachings herein. Alternatively, the construction of more specialized apparatus to perform the required method steps may be appropriate.

In addition, embodiments of the present invention may be implemented as a computer program(s) or software. It would be apparent to a person skilled in the art that the individual steps of the methods described herein may be put into effect by computer code. The computer program is not intended to be limited to any particular programming language and implementation thereof. A variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention. Furthermore, one or more of the steps of the computer program may be performed in parallel rather than sequentially.

Such a computer program may be stored on any computer readable medium. The computer readable medium may comprise storage devices such as magnetic or optical disks, memory chips, flash drives, memory sticks, or other storage devices suitable for interfacing with a general-purpose computer. The computer readable medium may also comprise a hard-wired medium such as the Internet system, or a wireless medium such as an IEEE 802.11 LAN. The computer program when loaded and executed on such a general-purpose computer effectively results in a system that implements one or more methods described herein.

### Glossary

A Glossary of terminology used hereinafter is set forth in Table 1.

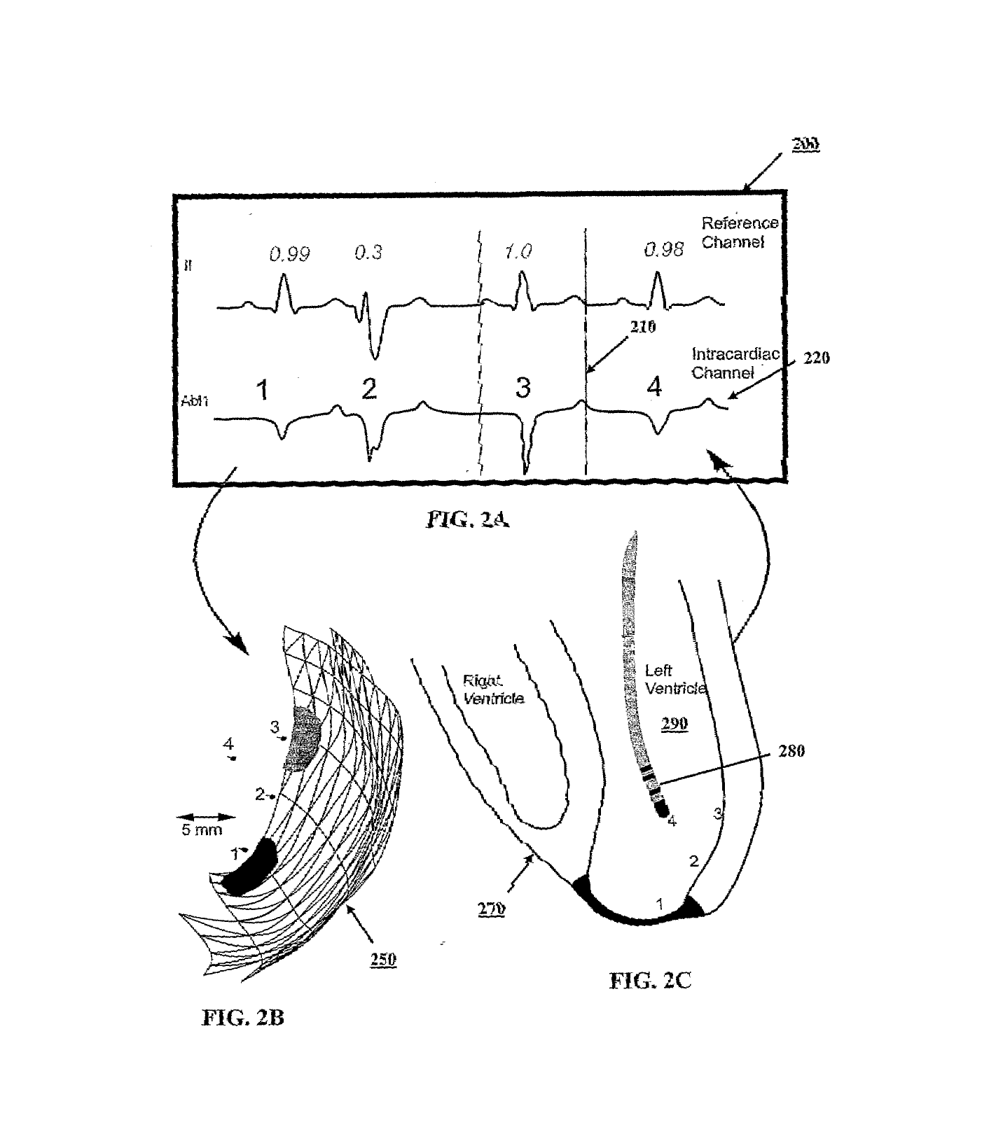

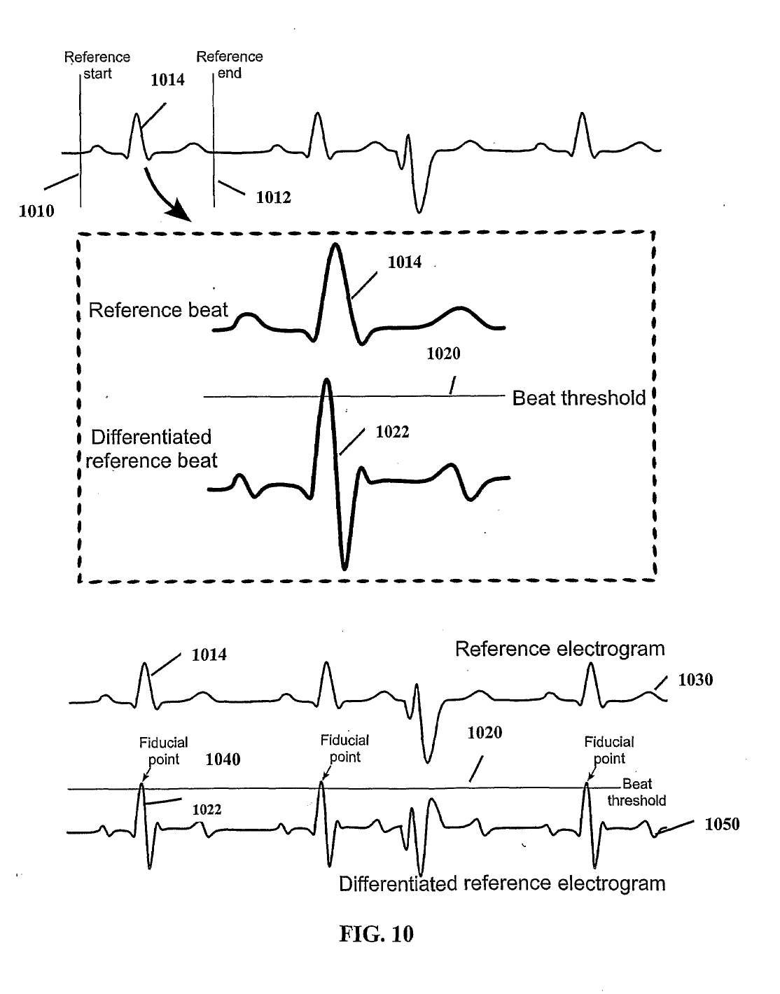

TABLE 1

| Term | Definition/Description |
|---|---|
| Beat | A single cardiac activation. The electrical and |
| | physiological changes that occur during the activation |
| | can be recorded in multiple channels. The time that a |
| | beat occurs is recorded in the analysed data file and is |
| | called the fiducial point for that beat. |
| Bipolar | An electrogram that has been recorded from two closely |
| Electrogram | spaced electrodes at the point of interest. |
| Catheter | An electrophysiology catheter; a medical device designed |
| | to record electrical signals from the heart comprising a |
| | long body with several electrodes at the distal tip. |
| Channel | Data collected from a single sensor, which is usually an |
| | electrode, but may also be a pressure monitor, etc. |
| | Therefore, each ECG lead is considered to be a channel |
| | and the channel ECG V1 refers to all of the data that has |
| | been collected from that electrode. |
| Differentiated | Differentiation is usually applied to a unipolar |
| Electrogram | electrogram so that the final data series shows the |
| | change in voltage rather than the absolute voltage. |
| | Therefore the differentiated electrogram is the slope of |
| | the raw electrogram at any given time point. See example |
| | 1050 in FIG. 10. |
| Electrogram | A recording taken from an electrode during a given time |
| | period. An electrogram is hence a subset of a channel |
| | (i.e. the segment of ECG V1 during the 3^{rd} beat would be |
| | considered an electrogram). See example 1030 in FIG. 10 |
| Fiducial | The fiducial point is an index into the recorded data |
| point | giving the exact time point (in milliseconds) that a beat |
| | occurred. See example 1040 in FIG. 10. |
| Mapping | A catheter that is usually capable of being flexed and |
| catheter | deflexed by operating a device within the handle. This |
| | catheter can therefore be moved to a variety of different |
| | locations within the heart by flexing and deflexing the |
| | catheter as needed. The mapping catheter also has an |
| | associated system designed to measure the exact location |
| | of the catheter tip in 3-dimensional space. In one |
| | embodiment, the mapping catheter typically has four |
| | electrodes, with the most distal electrode location at |
| | the extreme tip of the catheter. The other 3 electrodes |
| | are arranged as rings proximal to the distal electrode |
| | (see reference numeral 280 on FIG. 2C). Therefore four |
| | mapping channels can be recorded from the mapping |
| | catheter. |
| Real time | Analysis that is performed while data is still being |
| analysis | acquired. A limited subset of critical analytical tasks |
| | is performed to allow the computational system to keep |
| | pace with the rate of data acquisition. Real-time |
| | analysis allows the operator to view a substrate map, |
| | which includes data from all of the beats that have been |
| | recorded to that time without having to wait for a |
| | separate data analysis step. The number of different |
| | analytical steps that can be performed on each beat is |
| | dependent on the abilities (i.e. processing power) of the |
| | computational equipment that is used. |
| Reference | The data from this beat is compared with all of the other |
| beat | beats within the recorded data. A correlation process |
| | allows similar beats occurring throughout the recording |
| | to be grouped for analysis. Multiple reference beats can |
| | be analysed with the results from each analysis being |
| | stored separately. See example 1014 in FIG. 10. |
| Reference | A catheter that remains in one location within the heart |
| catheter | for the duration of the procedure (eg. the high right |
| | atrium or the right ventricular apex). Electrograms |
| | recorded from this catheter should therefore be the same |
| | from one beat to the next as long as the heart remains in |
| | the same rhythm. |
| Reference | A reference channel can be used by the embodiments of |
| channel | the invention to either mark the time of activations |
| | (i.e. find the fiducial points of beats within the |
| | recordings) or to compare different beats with each |
| | other. Multiple reference channels can be chosen and the |
| | analysis results from each channel are stored in |
| | different values (eg. some ectopic beats may only be |
| | discernible in a specific ECG channel). |
| Unipolar | An electrogram that has been recorded from a single |
| Electrogram | electrode at the point of interest. |

### Overview

Embodiments of the invention provide automated processing of intracardiac electrophysiological data. The embodiments of the invention can rapidly analyse recorded electrogram data using an index of temporal locations to locate electrophysiological features suggestive of an abnormality. For example, this may identify critical sites responsible for cardiac arrhythmias. As set forth in greater detail herein after, electrogram data and corresponding spatial location data of an electrode recording the electrogram data are recorded. The recorded electrogram data comprises a number of beats. At least one reference channel containing a reference beat is defined for determining temporal locations and against which beats of the recorded electrogram data are compared. An index of the temporal locations and other information of the beats within the recorded electrogram data is created. At least one electrophysiological feature of the recorded electrogram data suggestive of a physiological condition is analysed in real time. An updated index is provided wherein the other information comprises results of the analysis.

The ability to accurately identify ventricular scarring is important as ventricular tachycardia (VT) re-entrant circuits are often located in the border zone surrounding scarred myocardium and many of the successful strategies to treat VT depend on accurate scar localization. Myocardial scarring acts as an obstruction to the flow of the electrical activity within the cardiac chamber. During mapping procedures for atrial flutter or fibrillation, areas of myocardial scar must be located as those areas may act as a fixed obstruction around which the abnormal electrical activity travels in a circular fashion. Accurate scar localisation is therefore of critical importance during radiofrequency ablation procedures. Scar localisation is also useful in other situations, including choosing appropriate locations to implant pacing leads, and the diagnosis of conditions such as Arrhythmogenic Right Ventricular Dysplasia in which large portions of the myocardium are replaced with fat. The location of a scar may be identified using one of many electrophysiological systems that can determine the 3-dimensional (3D) location of the catheter within the cardiac chamber and therefore identify the location from which each recorded beat has been recorded. Conventionally, such maps have typically been constructed sequentially with the operator inspecting a single beat and either adding the beat to the map or recording another more suitable beat from the same location. This technique is labour intensive leading to further increases in the duration of these long and complicated procedures.

One embodiment of the invention provides a technique that can rapidly create spatial maps showing the location of myocardial scarring and other electrophysiological features (e.g., the presence of diastolic electrical activity or electrical activity originating from other cardiac chambers such as the atria) that may indicate critical sites responsible for cardiac arrhythmias. The spatial location of each beat can be added to the indexed data for that beat. This allows the automated mapping program to display spatial maps of electrogram characteristics and one specific application is the construction of substrate (‘Scar’) maps. Many of the regions of abnormal conduction that require treatment are located near scarred myocardium and for this reason it is valuable for the clinician to be able to determine the location of myocardial scarring. An accurate map can be constructed with minimal user input allowing the operator to concentrate on other aspects of the procedure. This system is also capable of automatically marking the time of activation at different regions of the chamber to allow an arrhythmia to be mapped rapidly. Physiological data derived from other catheter mounted sensors, such as pressure or chemical concentrations, can also be added to the index data for each beat allowing this information to be incorporated into 3-dimensional maps.

In another embodiment of the invention, the analysis results are used to trigger an alarm. This may be done alone or in addition to generating the substrate map. The real-time analysis results may be used in other practical applications.

A paradigm is disclosed hereinafter for recording, viewing and analysing the data that is collected during an electrophysiology study. Previously, data was recorded sequentially and the main way to access the recorded data was through a time-based index. The embodiments of the invention may be implemented in software that acts as a digital assistant to identify and catalogue every beat that occurs during the study. These beats can then be selected on a variety of criteria such as spatial location, morphology (eg. ‘sinus beats’ or ‘ventricular ectopics’), electrogram voltage or slope, and time. Benefits of this technology include the ability to collect high quality data quickly. Further, the results can be compiled and displayed in the form of spatial maps to show the locations of abnormal areas within the heart.

An automated map may be constructed using the following steps: electrogram and location data recording, data synthesis, beat detection, beat classification, electrogram analysis, substrate map review and substrate map enhancement. These topics are described hereinafter, but the steps do not need to be performed in this specific order in practice (eg. beat detection can be repeated after the substrate map has been created if the user detects a problem with the initial choice of reference beat).

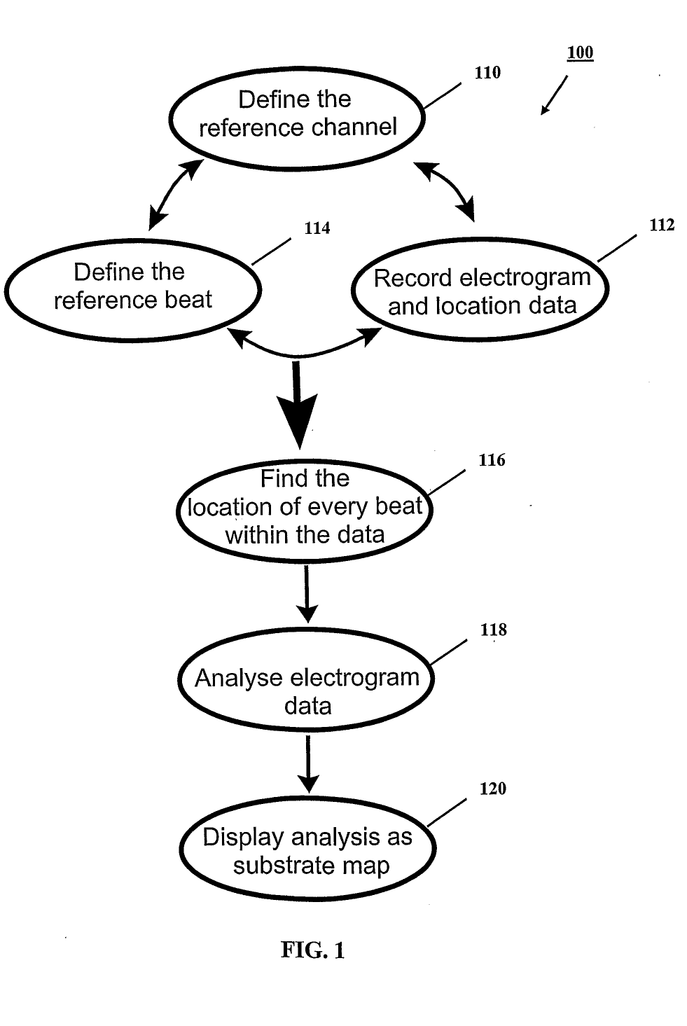

FIG. 1 is a high-level flow diagram illustrating this process 100 of creating and displaying substrate maps in accordance with an embodiment of the invention. In step 110, a reference channel is defined. Processing continues in a bi-directional manner between step 110 and each of steps 112 and 114. Similarly, processing may flow in a bi-directional manner between steps 112 and 114. In step 112, electrogram and corresponding location data of the electrode(s) relative to the heart are recorded. The method uses a cardiac electrophysiology system that is capable of recording both electrogram data and the 3-dimensional locations from which this data is recorded. For example, a variety of electrophysiology systems are available that can calculate the 3-D location of the catheter tip using magnetic (eg. NOGA™ or CARTO™ systems), ultrasound (eg. RPM™), or impedance (eg. Navex™, EnSite3000™ or LocaLisa™) monitoring technologies. In step 114, a reference beat is defined. From steps 110, 112, 114, processing continues at step 116. In step 116, the location (expressed as a time point) of every beat within the recorded data is found. In step 118, the electrogram data is analysed in real time. The foregoing steps constitute one embodiment of the invention. The analysis results may be used, for example, to trigger an alarm. In yet another embodiment, a substrate map may be generated. Thus, in accordance with this further embodiment of the invention, in step 120, the analysis results of step 118 are displayed as a substrate map. The method in accordance with this embodiment allows a 3-D representation of the chamber of interest to be created with minimal user intervention allowing rapid and accurate localisation of scarred and diseased myocardium. This substrate map can then be reviewed and enhanced by adding further information as required. This can also be used to characterise the spread of activation throughout the myocardium to create an activation map. The system then processes any stored background tasks while waiting for further user input or recorded data to analyse. This method may be implemented in one embodiment as a computer program.

FIG. 2A shows an electrogram review window containing a reference channel 210 and an intracardiac channel 220 illustrating four beats (1, 2, 3, 4). The corresponding recording locations (1, 2, 3, 4) for the beats are graphically shown in FIG. 2C. A heart 270 is depicted in FIG. 2C with a catheter 280 disposed with the left ventricle 290 for beat 4. The third beat is defined as the reference beat 210. The correlations (0.99, 0.3, 1.0, and 0.98) of the measured beats 220 with the reference beats 210 are displayed in light grey above each beat. The automated map 250 that has been created with the electrogram data is shown in FIG. 2B.

During the first beat, the mapping catheter 280 shows a small deflection indicating scarred myocardium in FIG. 2C and so the corresponding region of the substrate map 250 is marked with a (dark grey) hatched pattern to indicate scarred myocardium in FIG. 2B. This beat has a high correlation of 0.99. The mapping catheter 280 is moved to another location during beat 2 in FIG. 2C, but this beat is a premature ventricular complex (correlation only 0.3 with the reference beat) and so no data from this beat is added to the substrate map 250. When the third beat is recorded, the catheter 280 has been moved to another location (position 3) in FIG. 2C, which shows a large deflection, and this area is represented as normal with a light grey pattern on the substrate map 250 in FIG. 2B. This beat has a correlation of 1.0. The catheter 280 is then moved to position 4 when the fourth beat is recorded in FIG. 2C, but the program does not add this data to the substrate map 250 because the catheter 280 is too far away (i.e. farther than the interpolation distance of 5 mm) from the surface of the substrate map 250 in FIG. 2B.

Thus, the embodiments of the invention enable a clinician to access massive amounts of recorded data to identify any abnormal regions within the heart responsible for cardiac arrhythmias. In particular, the method can record and automatically analyse sections or the entirety of a cardiac procedure. The method may be implemented using a computer program that acts as a digital assistant to automatically locate and then index each beat within the recorded segments. This allows the clinician to view tracings of beats fitting specific criteria (eg. the presence of double potentials). Automated mapping in this manner allows a substrate map to be created with minimal user intervention. This allows a faster and more successful procedure to be performed. Further, reducing the procedural time has a major impact on patient wellbeing as this reduces the radiation dose, risk of complication, and discomfort to the patient. Reducing the procedure time also has clear benefits for the operator and hospital allowing more patients to be treated with the limited staff and equipment available. If an automated substrate map is constructed with serendipitously acquired electrogram data, the operator can then use the time that has been saved to improve further the mapping accuracy.

### Display Windows

To display data and collect input from the user, a computer program in accordance with an embodiment of the invention uses ‘windows’ or graphical displays listed in Table 2.

TABLE 2

| Term | Definition/Description |
|---|---|
| Electrogram | This display shows the reference electrogram as |
| window | well as the raw and differentiated mapping catheter |
| | electrograms. The user can navigate to any portion |
| | of the recorded electrograms by using the slider |
| | controls. The electrograms can be displayed at a |
| | variety of magnification levels (‘sweep speeds’). |
| Automated | This display shows the cardiac chamber as a 3- |
| map window | dimensional model. The colours at each point on |
| | the map represent the analysis results of |
| | electrograms recorded from that point. |
| Zoomed | This displays the mapping electrograms that were |
| electrogram | recorded from a user selected region of interest. The |
| window | data is represented in two-dimensions and each beat |
| | is shown as a scaled electrogram. This view allows |
| | the user to remove electrogram data points that have |
| | incorrect or artefactual data from the substrate map |
| | by clicking on them. |

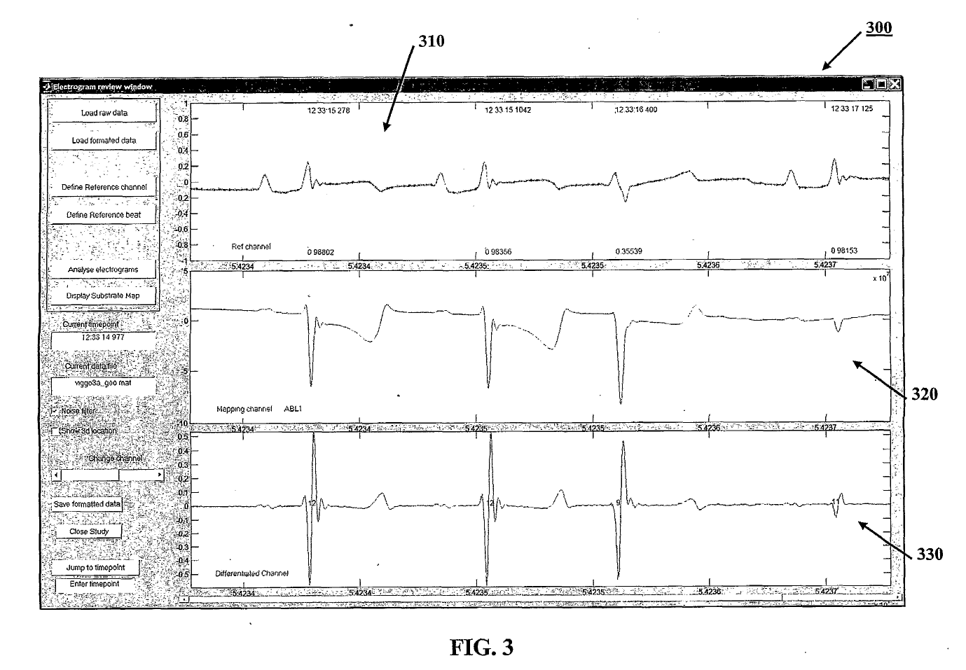

FIG. 3 illustrates an electrogram review window 300. The reference channel 310 (in this case ECG lead II) is shown in the top panel. Each beat has been identified, and the correlation between the beat and the reference beat is shown at the bottom of the panel near the dashed vertical line through the respective beat. The third beat is a premature ventricular complex, and the system shows that it has a low correlation (0.356) with the reference beat. The signals from the mapping catheter 320 are shown in the middle panel (in this case the distal unipolar electrogram). The lowest panel has the differentiated mapping signal 330, which represents the instantaneous slope in the mapping channel.

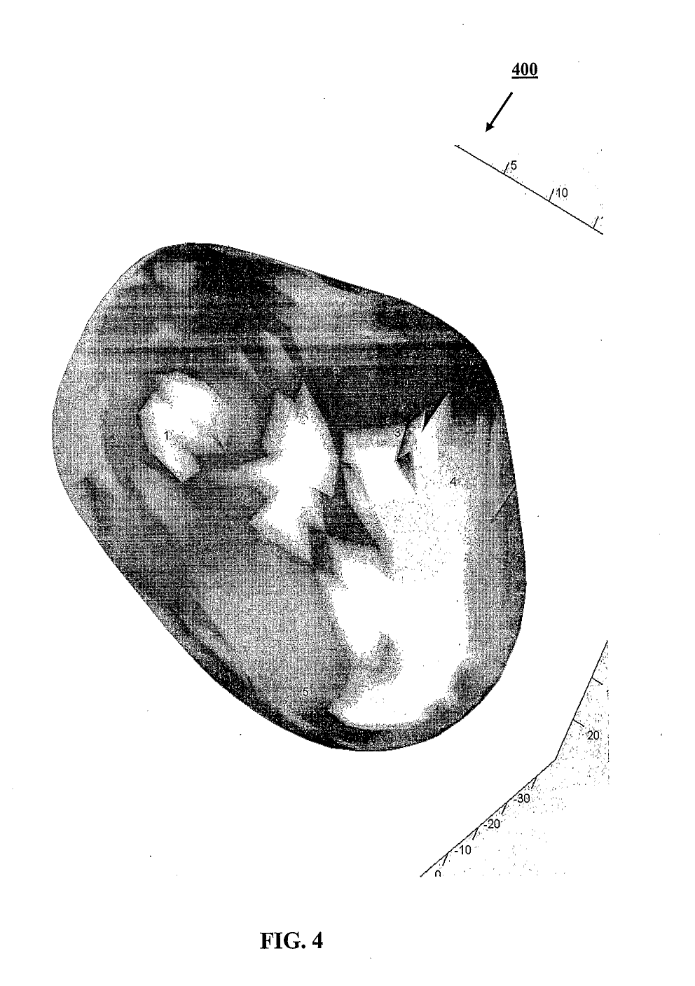

FIG. 4 is a plot illustrating an automated substrate map window 400, which is a 3-dimensional representation of the chamber of interest (in this example, the left ventricle). Areas that have not been analysed using the mapping catheter are represented with light grey fill. Normal myocardium is represented by dark grey fill and abnormal areas are represented by the white fill. Catheter locations 1-5 are shown (numbered) in the drawing.

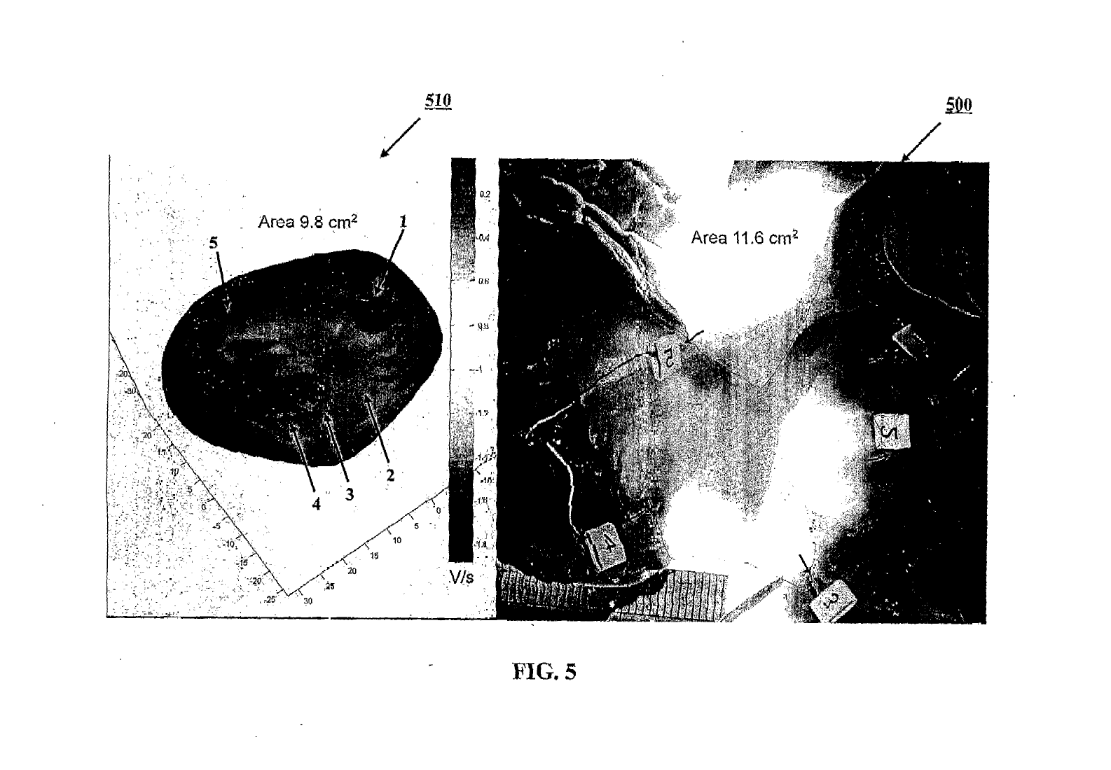

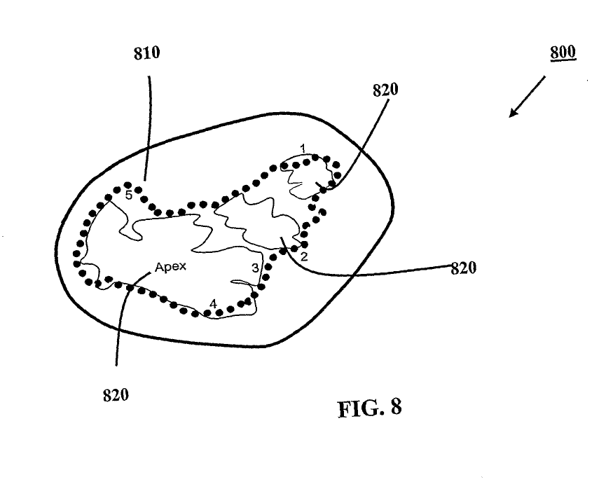

FIG. 5 shows an automated 3-dimensional substrate map 510 (on the left), for example, that has been constructed from a sheep's heart 500 (on the right) using the maximal negative slope of the roving catheter distal electrode. A threshold value of greater than −0.25 V/s is set, so areas with slopes less than −0.25 V/s are shown as dark grey (i.e. are considered to not have transmural scar) and unmapped areas are shown as light grey in map 510. This area of the heart 500 (right panel) has been transilluminated to show the transmural scar. The gross specimen shows the endocardium viewed from within the chamber and has hence been projected in reverse to give the same spatial orientation as the substrate map 510, which shows the endocardial surface viewed from outside the chamber. The location of the marker needles (1, 2, 3, 4, 5) is shown in both panels with the exact insertion points given by the small black arrows in the left panel. The boundary lines used for area measurements and the respective areas are given in the two panels. A diagrammatic depiction of the map 510 of FIG. 5 is shown in black and white as the map 800 in FIG. 8. The dotted line 810 represents the outer border of the myocardial scar identified using detailed histological analysis. The automated mapping program identifies the three abnormal regions 820 which are accurately located within the scar. The numbers 1-5 in FIGS. 5 and 8 represent the location of the five marker needles.

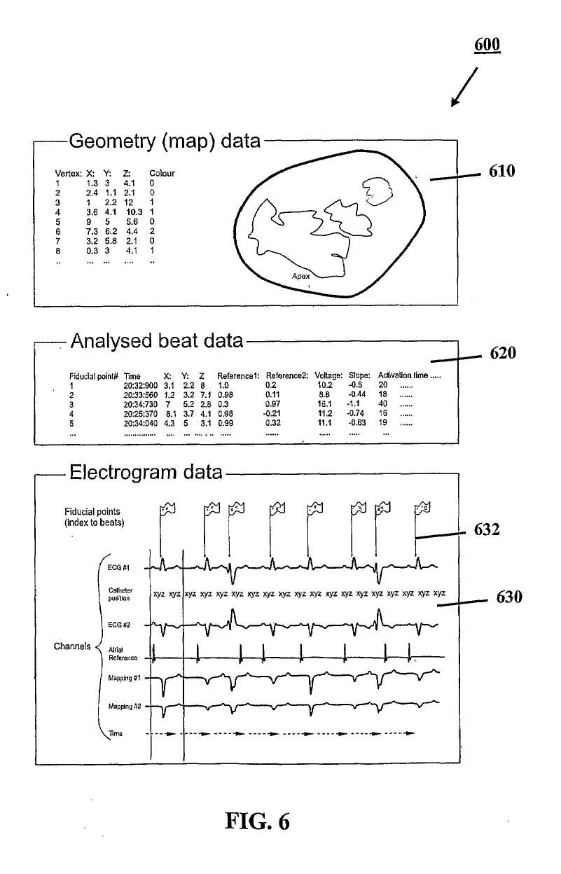

FIG. 6 shows three main forms of data 600 collected and used by the automated mapping system: geometry (map) data 610, analysed beat data 620, and electrogram data 630. The list of fiducial points 632 (index to beats) can be visualised as pins placed on the recorded data 630, denoting the time that each beat occurred. The information collected by the system is added to these pins (see lowest panel) to provide further classification of each beat. The electrogram data 630 includes ECG #1 channel, catheter position data (X, Y, Z), ECG #2 channel, an atrial reference channel, a mapping #1 channel, a mapping #2 channel, and time. Please refer to the above glossary for an explanation of these different channels. Typical analysed data available for each beat 620 is shown in the middle panel. The analysed data, in this example, includes the fiducial point number, the time, the location (X, Y, Z), reference 1, reference 2, voltage, slope and activation time.

In this example, a beat originating from the sinus node is chosen as the reference beat 1. There are also frequent extra beats originating from within the ventricle (“Ventricular Ectopic Beats” or VEBs) and one of these VEBs is chosen as reference beat 2. The VEBs can be recognised on the ECG channels as having a different morphology (shape) and a greater amplitude and duration. The analysed data recorded in the Reference 1 column therefore shows a high correlation (>0.95) if the analysed beat resembles reference beat 1 indicating that the analysed beat also originates from the sinus node. The analysed data recorded in the Reference 2 column would correspondingly show a high correlation if the analysed beat resembles reference beat 2 indicating that the beat originated from the ventricle. In the top panel, the geometry data 610 is shown. The geometry data 610 includes the vertex number, the location (X, Y, Z) and colour. The main data 610 required to construct the geometry are the exact location of each point within the geometry and the colour that each of these points should be given.

### Data Recording (AL1)

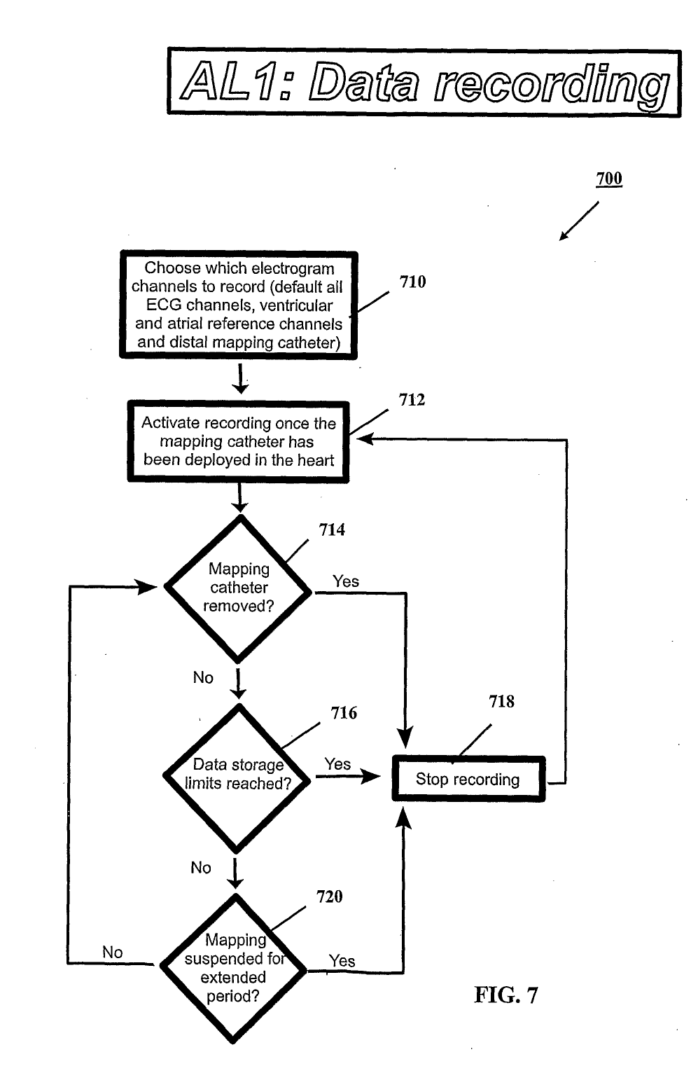

Electrograms are recorded from intracardiac catheters and surface ECG patches. The number of electrogram channels that are recorded is variable, depending on the system capabilities (i.e. available memory and storage space) and number of intracardiac catheters that are available in a given case. At a minimum, a surface ECG channel and the intracardiac electrogram from the roving catheter tip are recorded. The 3-D location of the roving catheter tip must also be recorded. If present, the surface ECG leads and intracardiac signals from the fixed catheters in the atrium, coronary sinus, and ventricle may be recorded. The intracardiac electrograms may be recorded as both unipolar and bipolar signals. The data may be recorded at a specified sampling rate, e.g. the native sampling rate of the electrophysiology system (typically 1000 samples/second, although other sampling rates may be practiced). Filtering may used while recording the unipolar electrograms, but preferably minimal filtering is involved. If the mapping catheter is removed from the cardiac chamber or mapping is suspended for extended periods of time, the system stops recording data. The details of this process are shown in FIG. 7.

FIG. 7 is a flow diagram illustrating the process of data recording (AL1) 700 in accordance with the embodiment of the invention. The process 700 commences at step 710. In step 710, the electrogram channels to record are chosen. The channels to record may vary depending upon the application. In one embodiment, the default channels are all ECG channels, ventricular and atrial reference channels, and the distal mapping catheter. In step 712, once the mapping catheter has been deployed in the heart, the recording is activated. In decision step 714, a check is made to determine if the mapping catheter has been removed. If step 714 returns true (Yes), processing continues at step 718, in which recording stops. Processing then continues at step 712. Otherwise, if decision step 714 returns false (No), processing continues at step 716. In decision step 716, a check is made to determine if the data storage limits for the recordings have been reached. If step 716 returns true (Yes), processing continues at step 718. Otherwise, if step 716 returns false (No), processing continues at step 720. In decision step 720, a check is made to determine if the mapping has been suspended for an extended period. If step 720 returns true (Yes), processing continues at step 718. Otherwise, if step 720 returns false (No), processing continues at step 714.

### Data Synthesis (AL2)

A differentiated electrogram signal is constructed from each unipolar electrogram. The differentiated signal may be constructed by subtracting a data point at a specified time, e.g. 2 milliseconds, before the time zero point from the data point at another specified time, e.g. 2 milliseconds, after the time zero point. This value (measured in Volts) is then divided by the number of steps between the two measurements, e.g. 4 steps, and multiplied by the sampling rate, so that the differentiated electrogram signal has units of Volts per second. This methodology produces a relatively noise free differentiated electrogram and is computationally efficient (i.e. requires minimal computation as the methodology can be utilised within the vector processing routines of modern CPUs) allowing the methodology to be performed in real time.

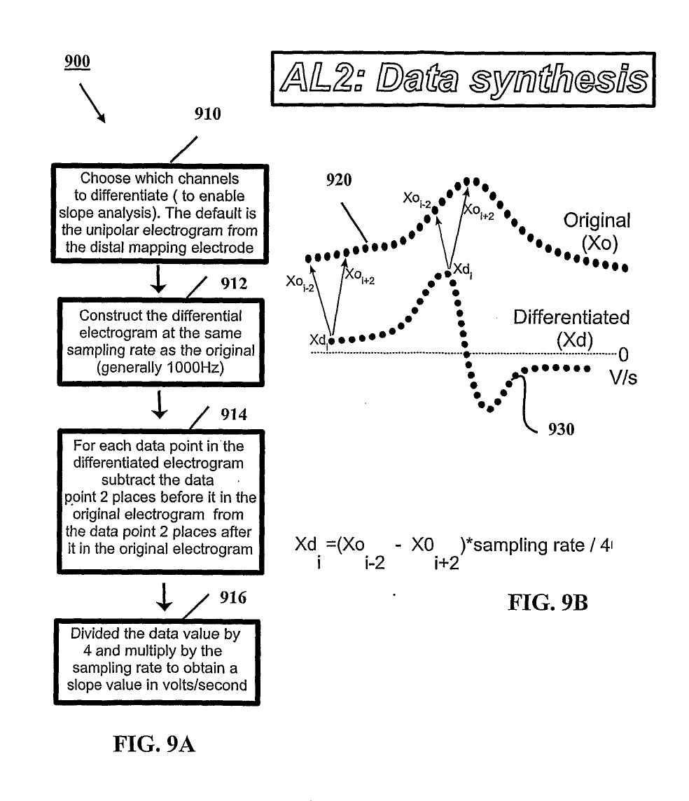

FIG. 9A is a flow diagram illustrating the process of data synthesising (AL2) 900 in accordance with the embodiment of the invention. FIG. 9B is a plot showing the original signal Xo 920 and the resulting differentiated signal Xd 930, which is calculated using the following formula:

Xdᵢ=(Xoᵢ₋₂−Xₒᵢ₊₂)*sampling rate/number of steps between measurements.

In FIG. 9B, two arrows extend from each of two points Xd in the differentiated signal Xd 930 to the points xoᵢ₋₂ and xoᵢ₊₂ in the original signal Xo 920.

The process 900 commences in step 910. In step 910, the channels to differentiate are chosen. This is done to enable slope analysis. The default may be the unipolar electrogram from the distal mapping electrode. This provides the original signal 920. In step 912, the differential electrogram is constructed at the same sampling rate as the original signal (generally, 1000 Hz, but other sampling rates may be practiced). In step 914, for each data point xdᵢ in the differentiated electrogram, a data point two places xoᵢ₋₂ in the original electrogram before the corresponding data point is subtracted from the data point two places xoᵢ₊₂ after the corresponding data point. In step 916, the data value xdᵢ is divided by four and multiplied by the sampling rate to obtain a slope value in volts/second.

Although the differentiated electrogram has been calculated over a four-data-step interval in these examples, a variety of intervals can be used depending on user preference, the characteristics of the data being analysed (e.g. sampling rate and the presence of noise within the recorded data), and the limitations of the computational hardware. For example, the data interval could be increased to 8 by subtracting four places Xoᵢ₋₄ in the original electrogram before the corresponding data point is subtracted from the data point four places Xoᵢ₊₄ after the corresponding data point (corresponding formula: Xdᵢ=Xoᵢ₊₄−Xoᵢ₋₄*sampling rate/8). By increasing the data interval to 8, the operator would obtain a differentiated electrogram that has less noise (random variations), but lower temporal resolution would result (decreased ability to differentiate between two events that occurred at similar times). Alternatively, if the operator is able to increase the data sampling rate to 2000 samples a second, the operator is able to use an 8-data-interval differentiated electrogram without losing temporal resolution. However, increasing the data sampling rate increases data storage and computational requirements.

### Beat Detection (AL3)

In beat detection, the operator identifies a beat to be used as a reference against which the other beats within the recorded data are compared. FIG. 10 illustrates a beat 1014 of an electrogram between a reference start time 1010 and a reference end time 1012 that can be used as a reference beat 1014. This is typically a sinus beat, but could also be a paced or ectopic beat. The chosen beat can be replaced at a later stage if the operator wants to construct additional substrate maps (eg. a map constructed from ectopic beat data only) or correct the current substrate map (eg. an incorrect beat was chosen as the initial reference beat). The reference beat may be chosen manually by defining the start and end (1010, 1012) of the beat with a mouse or another input device. Optionally, an initial reference beat may be chosen by the system using predetermined criteria. These criteria may include:

1. Most common beat morphology (eg. 10 beats at the start of the recorded segment may be chosen and correlated with each other, and the beat with the highest mean correlation to all of the others is chosen); and

2. Comparison with a global reference beat (eg. the first 10 beats are compared with a standard reference beat, and the closest match is used as the reference beat for this study)

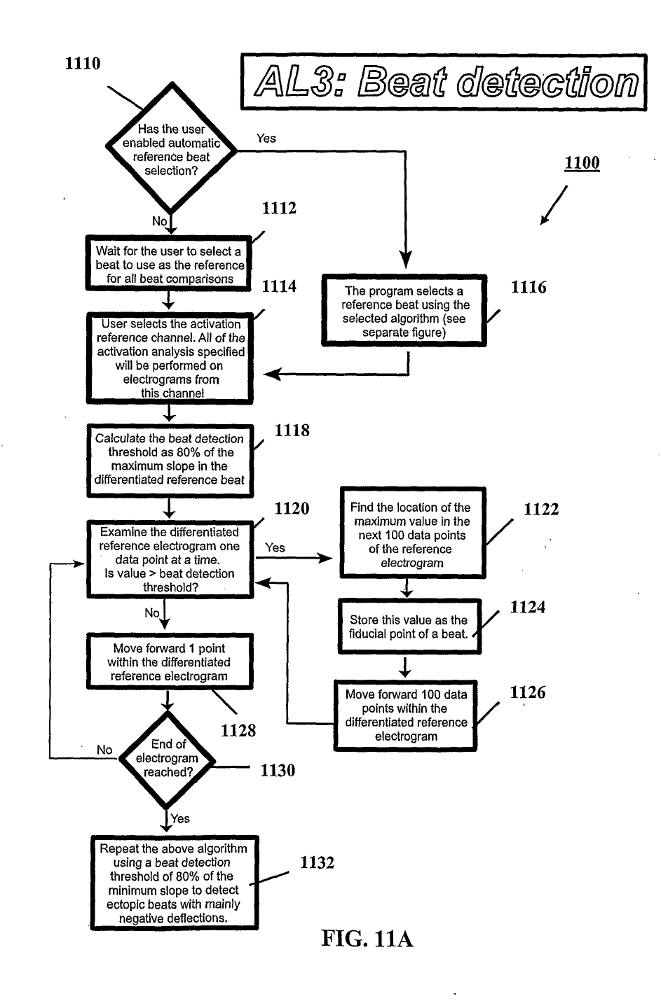

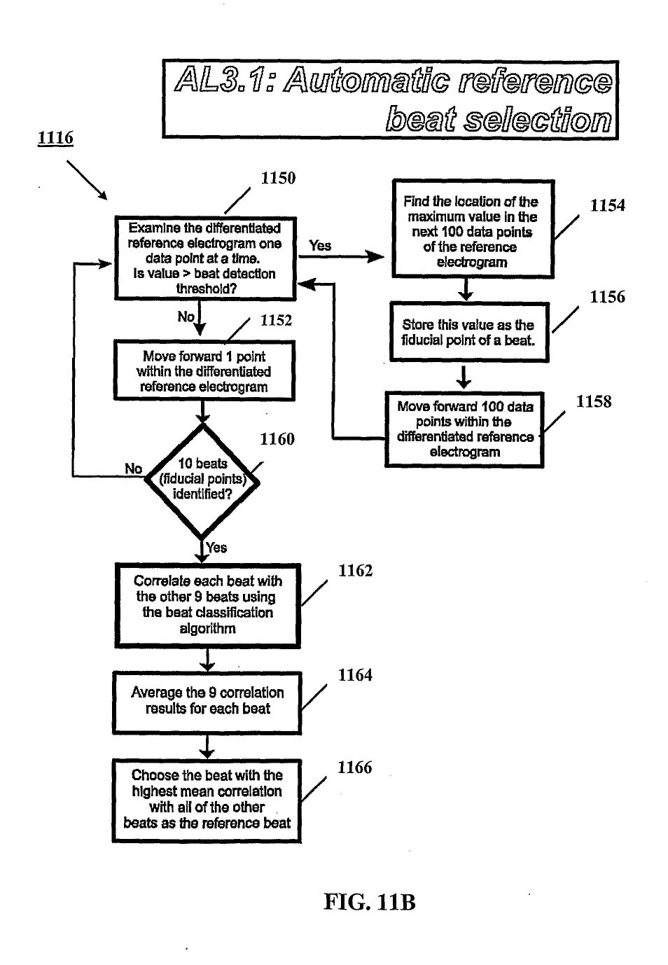

The operator may chose which of these criteria (singly or in combination) to use by default for subsequent cases. FIG. 11B provides further details.

A reference channel that shows clear activation of the chamber of interest is then chosen. This may be an intracardiac or surface electrogram. The reference channel records data from the same location throughout the study segment that is being analysed. The reference channel is used to mark the activation time for each beat. The reference channel is also used to compare beats to a reference beat; the reference channel can be used to differentiate between the baseline cardiac rhythm and any ectopic beats. In some cases, two different reference channels must be specified—one to mark the activation time and the second to differentiate beats originating from different locations. This is particularly likely when mapping the atria as an atrial reference catheter is required to determine correctly the atrial activation time, but an atrial catheter alone could not be used to differentiate between sinus and ectopic beats.

Referring to FIG. 10, the timing of each beat within the data file is calculated from the reference electrogram channel. A beat threshold value 1020 is calculated by finding the maximum value in the differentiated reference beat 1022 (i.e. between times 1010 and 1012) and multiplying this value by 0.8 to obtain the beat threshold 1020. The differentiated reference channel is analysed, and if a data point exceeds the threshold 1020, the maximum value in the next 100 data points is found and stored as a fiducial point 1040 within the analysed data index file. Using this methodology, a list of the location of all of the beats within the data file can be compiled efficiently. This methodology has the added advantage that the methodology is unlikely to produce false positive beat detections due to ‘T’ waves as even large ‘T’ waves tend to have small slopes. FIG. 11A provides further details of this process.

FIG. 11A illustrates a process of detecting beats (AL3) 1100, in which processing commences in step 1110. In decision step 1110, a check is made to determine if the user has enabled automatic reference beat selection. If step 1110 returns true (Yes), processing continues at step 1116. In step 1116, a reference beat is selected using an automatic reference beat selection process shown in FIG. 11B, which is described in detail hereinafter. Processing then continues at step 1114. Otherwise, if step 1110 returns false (No), processing continues at step 1112. In step 1112, the system waits for the operator to select a beat to use as the reference for all beat comparisons. In step 1114, the operator selects the activation reference channel. All of the activation analysis specified is performed on electrograms from this channel.

In step 1118, the beat. detection threshold is calculated as eighty percent (80%) of the maximum slope in the differentiated reference beat. Alternative threshold values may be chosen depending on the characteristics of the recorded data and user preference. Setting the beat detection threshold to less than 80% of the maximum slope in the reference beat (eg. 70%) increases the sensitivity of beat detection (i.e. fewer beats are missed), but decreases the specificity of beat detection (i.e. the algorithm may activate falsely and create a ‘false’ beat). Increasing the beat detection threshold has the opposite effect on the sensitivity and specificity. Alternatively, two or more reference channels may be used to mark the time of beats within the recorded data (eg. a beat is detected if the value exceeds a threshold value in two ECG channels). Using the automated mapping software, a large amount of data is gathered (i.e. potentially every beat that occurs during the procedure) and some of this data is redundant (i.e. electrograms may be recorded from the same location due to slow movement of the catheter). Therefore, theoretically a threshold that increases the specificity of data collection may be chosen, because the data redundancy ensures that the map is accurate even if the software discards some beats incorrectly.

Alternatively, beat identification may be performed using other physiological signals such as intraventricular pressure if this data is available. For example, the program may be set to identify the fiducial point at the time when the intraventricular pressure exceeded 30 mm Hg, indicating ventricular contraction.

In step 1120, the differentiated reference electrogram is examined one data point at a time to determine if its value is greater than the beat detection threshold. If step 1120 determines the value is greater than the threshold, processing continues at step 1122. In step 1122, the location of the maximum value in the next specified time interval, e.g. 100 ms, (i.e. 100 data points if the data sampling rate is 1000 samples per second) of the electrogram are found. The time interval of 100 milliseconds is chosen to prevent the algorithm from registering false activations within the same beat (i.e. counting the same beat twice because the beat has more than one large positive deflection). This time interval can therefore be defined as the blanking period for beat detection. Alternative time intervals (blanking periods) can be practiced, but large blanking periods (>200 ms) decrease the ability of the program to analyse properly fast cardiac rhythms as the next beat may fall within the blanking period.

In step 1124, this maximum value is stored as the fiducial point of a beat. In step 1126, the processing moves forward the same number, e.g. 100, data points within the differentiated reference electrogram. Processing then returns to step 1120. Otherwise, if step 1120 determines the value is not greater than the threshold, processing continues at step 1128. In step 1128, processing moves forward 1 data point within the differentiated reference electrogram. In decision step 1130, a check is made to determine if the end of the electrogram has been reached. If step 1130 returns false (No), processing continues at step 1120. Otherwise, if step 1130 returns true (Yes), processing continues at step 1132. In step 1132, the algorithm of steps 1110 to 1130 is repeated using a beat detection threshold of 80% of the minimum slope to detect the ectopic beats with mainly negative deflections.

FIG. 11B is a detailed flow diagram of the automatic reference beat selection process of step 1116 of FIG. 11A. Processing commences in step 1150. In step 1150, the differentiated reference electrogram is examined one data point at a time to determine if the value of the data point is greater than a beat detection threshold. If step 1150 determines that the value is greater than the threshold (Yes), processing continues at step 1154. In step 1154, the location of the maximum value in the next specified number, e.g. 100, data points of the reference electrogram are found. In step 1156, this maximum value is stored as the fiducial point of a beat. In step 1158, the process moves forward 100 data points with the differentiated reference electrogram. Processing continues at step 1150. Otherwise, if step 1150 determines that the data point value is not greater than the beat detection threshold (No), processing continues at step 1152. In step 1152, the process moves forward one data point within the differentiated reference electrogram.

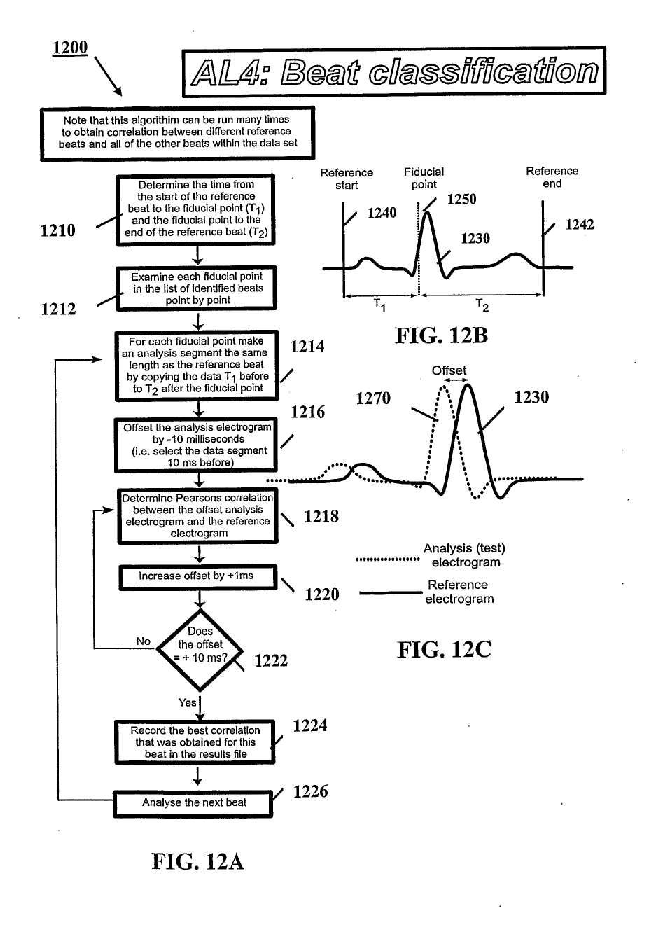

In decision step 1160, a check is made to determine if a predetermined number N, (e.g. N is an integer equal to ten) of beats (fiducial points) have been identified by step 1156. If decision step 1160 returns false (No), processing continues at step 1150. Otherwise, if decision step 1160 returns true (Yes), processing continues at step 1162. In step 1162, each beat is correlated with the other N−1 (e.g., nine) beats using a beat classification process 1200 of FIG. 12A, described hereinafter. In step 1164, the N−1 (nine) correlation results for each beat are averaged. In step 1166, the beat with the highest mean correlation with all of the other beats is chosen as the reference beat. Processing 1116 then terminates.

### Beat Classification (AL4)

Beat classification can be regarded as one of the many forms of analysis that is performed on every cardiac beat. However, it is worth considering beat classification as a separate entity, because the classification of a beat can be used to determine whether any further analysis must be performed on that beat during real-time operation. For example, beats that do not originate from the sinus node are unlikely to provide critical information for the real-time substrate map and can therefore be analysed by the program as a background process when time permits.

The correlation between each of the identified beats and the reference beat can be analysed using Pearson's correlation. Pearson's correlation is a pattern matching algorithm that can be performed using a variety of computationally efficient algorithms (eg. see J C Chiang, J M Jenkins and L A DiCarlo, “Digital Signal Processing Chip Implementation for Detection and Analysis of Intracardiac Electrograms”, Pacing and Clinical Electrophysiology, August 1994, Volume 17, Pages 1373-1379). Electrograms may also be compared with each other using other methods including Spearman's rank correlation, which has the advantage of being less sensitive to a single aberrant value. Neural networks may also be constructed and trained to differentiate between different beats. Alternatively, an expert system may be used to determine the origin of a particular beat from the electrogram characteristics. All of these alternatives to the Pearson's correlation require more computational steps to be performed to obtain a result. These alternatives may therefore be useful in situations where computational limits are less stringent, such as for offline analysis after the procedure or for real time analysis is performed on powerful computer hardware. The reference channel during the predetermined, e.g., 500 millisecond, time segment of each beat is compared with a corresponding, e.g. 500 millisecond, segment of the reference segment during the reference beat. The time of the maximal slope is used as the fiducial (time zero) point to line up the two segments. The correlation value may be performed a number of times, e.g. 20 times, with a specified, e.g. 1 millisecond, shifting of the two electrograms against each other to obtain the best possible correlation (‘lag shifting’). The technique of lag shifting ensures that errors in setting the timing of the two fiducial points do not cause a falsely low correlation, but does require extra computation and hence may not be possible on all systems during real time analysis. If the computer system is not sufficiently powerful in a computational sense to perform lag shifting in real time, the task may be saved and completed as a background process after the real-time display had been completed.

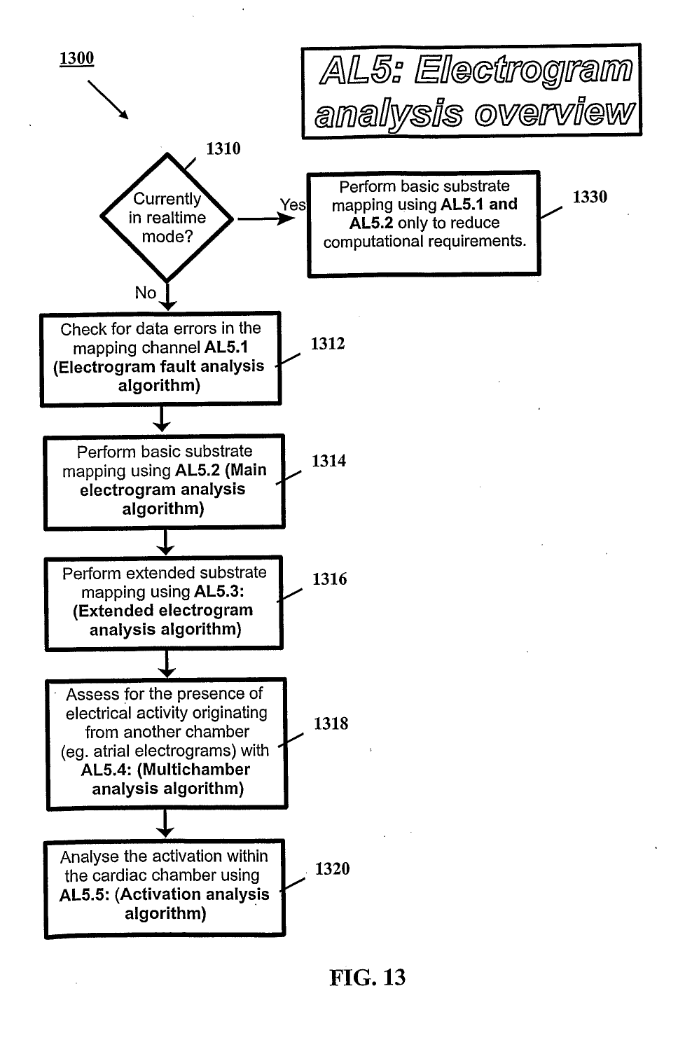

FIG. 12A is a flow diagram illustrating the process 1200 of beat classification, which is typically performed on the recorded data after a reference beat has been selected (as shown in FIGS. 11A and 11B) and before the data analysis step (as shown in FIG. 13). This process is also performed at step 1162 in FIG. 11B as part of the method for automatically choosing a reference beat. The process 1200 may be run a number of times to obtain correlations between different reference beats and all of the other identified beats within the recorded segment. Process 1200 commences in step 1210. In step 1210, the time T₁ from the start of a reference beat to a fiducial point and the time T₂ from the fiducial point to the end of the reference beat are determined. FIG. 12B depicts the times T₁ and T₂ for the reference beat 1230 with reference start and end times 1240 and 1242 and a fiducial point 1250. In step 1212, the computer program is instructed to examine every beat within the recorded data by performing instructions 1214 to 1226 beat by beat. Note that each beat is identified by its fiducial point, which is an index defining the exact time that the beat occurred. In step 1214, for each fiducial point, an analysis segment is made the same length as the reference beat by copying the data at the time T₁ before the fiducial point to the time T₂ after the fiducial point. In step 1216, the analysis electrogram segment is offset by a specified time, e.g. by −10 milliseconds (i.e., select the data segment 10 ms before). FIG. 12C shows an analysis (test) electrogram 1270 offset from a reference electrogram 1230.

In step 1218, the Pearsons correlation between the offset analysis electrogram 1270 and the reference electrogram 1230 is determined. In step 1220, the offset is increased by a specified amount, e.g. by +1 ms. In decision step 1222, a check is made to determine if the offset equals a predetermined threshold, =+10 ms. If step 1222 returns false (No), processing continues at step 1218. Otherwise, if step 1222 returns true (Yes), processing continues at step 1224. In step 1224, the best correlation obtained for this beat is recorded in a results file. In step 1226, the next beat is selected for analysis. By this means, every beat within the recorded segment is compared with the reference beat. In this example, a lag shift of −10 milliseconds to +10 milliseconds is used, resulting in 21 correlation coefficients being calculated for each beat. The highest correlation value is recorded as the final correlation for each beat. The lag shift value can be modified by the user as required. A lower lag shift value requires significantly less computing time, but may cause falsely low correlation values due to slight timing variations between the two beats. A higher lag shift value gives a more robust correlation value, which is less influenced by timing errors but also requires more computing time. Processing then continues at step 1214.

### Electrogram Analysis Overview (AL5)

The recorded electrograms from the mapping catheter are analysed in real time. During real-time analysis, only the electrogram characteristics that have been shown to create the most accurate substrate map are analysed, leaving the other characteristics to be analysed as a background task. One electrogram characteristic that correlates well with the location of myocardial scarring is the minimum slope (i.e. minimum value within the differentiated unipolar electrogram signal recorded from that site) in the distal mapping catheter electrode. Other electrogram characteristics that may be subsequently analysed include:
1. The duration of the negative slope (measured by calculating the duration that the differentiated electrogram is below 20% of the minimal value for that beat) is calculated for unipolar electrograms only.
2. The maximum deflection (i.e. ‘size’) of the raw (undifferentiated signal) electrogram measured by subtracting the minimal value from the maximal value for that beat. The maximal deflection is calculated for unipolar and bipolar electrograms.

The computer program also examines the data representing the 3-dimensional location of the catheter for each beat. The catheter would be expected to move slightly during the beat due to cardiac contractions, and this movement would predominantly be towards or away from the centre of the chamber (on axis movement). The degree of movement perpendicular to this axis (off axis movement) may be calculated for each beat, and beats with an off-axis movement of greater than 5 mm may not be represented on the substrate map. The operator may change these cut-off values while reviewing the substrate map or change the default cut-off value before mapping commenced.

Each electrogram channel is also checked for any features suggestive of faults or artefacts during the analysed beat. Faulty data may be manifest as amplifier saturation or voltage spikes, for example. Amplifier saturation occurs when the recorded voltages exceed the operating range of the amplifier system and appear as flat sections at the top or bottom of the electrogram channel window. The computer program determines the maximum and minimum values within each electrogram channel. Sections of the electrogram are marked as saturated if the electrogram sections are within 1% of these values and the data point 10 milliseconds after them is also within 1% of these values. Portions of the electrogram where the voltage changes more than 2 mV in a millisecond are marked as containing spikes. The specific values for the above embodiment of the invention may be altered by the operator depending on the characteristics of the hardware (eg. electrogram amplifier, analog to digital converter and computing work station) that is being used.

The local activation time can be calculated by locating the time of the minimum slope in the unipolar electrogram from the distal electrode of the mapping catheter. The time of the fiducial point is then subtracted from this time to obtain the local activation time, for that beat, relative to the reference electrogram. The relative local activation time and the minimal slope are then written to the data file for the beat. This allows the user to exclude local activation times that have a shallow slope as these sites are likely to have poor contact with the myocardium.

The automated analysis program also examines the mapping electrogram for evidence of double potentials and diastolic potentials. These extended analysis algorithms are only performed on beats that show a high correlation with the reference beat. The program analyses the mapping electrogram 200 ms before and after local activation and finds all of the time points in which the slope is less than −0.25 V/s. If any of these time points are >40 ms from the local activation time, the time and slope of the secondary activation with the lowest slope (i.e. most negative) is recorded into the data file for that beat as a double potential. The computer program then analyses the mapping electrogram segment 200 ms after the fiducial point for this beat to 200 ms before the fiducial point for the next beat (diastolic segment). An 8 Hz high-pass filter may be applied to this segment to reduce the effect of atrial activity and T wave artefacts.

Data derived from the electrogram analysis may be stored as a computer variable for ease of access. This data can be arranged in a variety of formats; the data may be stored as a two-dimensional array or grid with each row representing the data from a single beat. Within each row, the first column represents the beat number (eg. beat 1 is the first recorded beat), followed by the time that the beat occurred and then the X, Y and Z location of the catheter tip when the beat was collected. The remaining columns may store the results of the various analysis methodologies detailed in the preceding paragraphs. See data 620 in FIG. 6. FIGS. 13-18 provide further details of this processing.

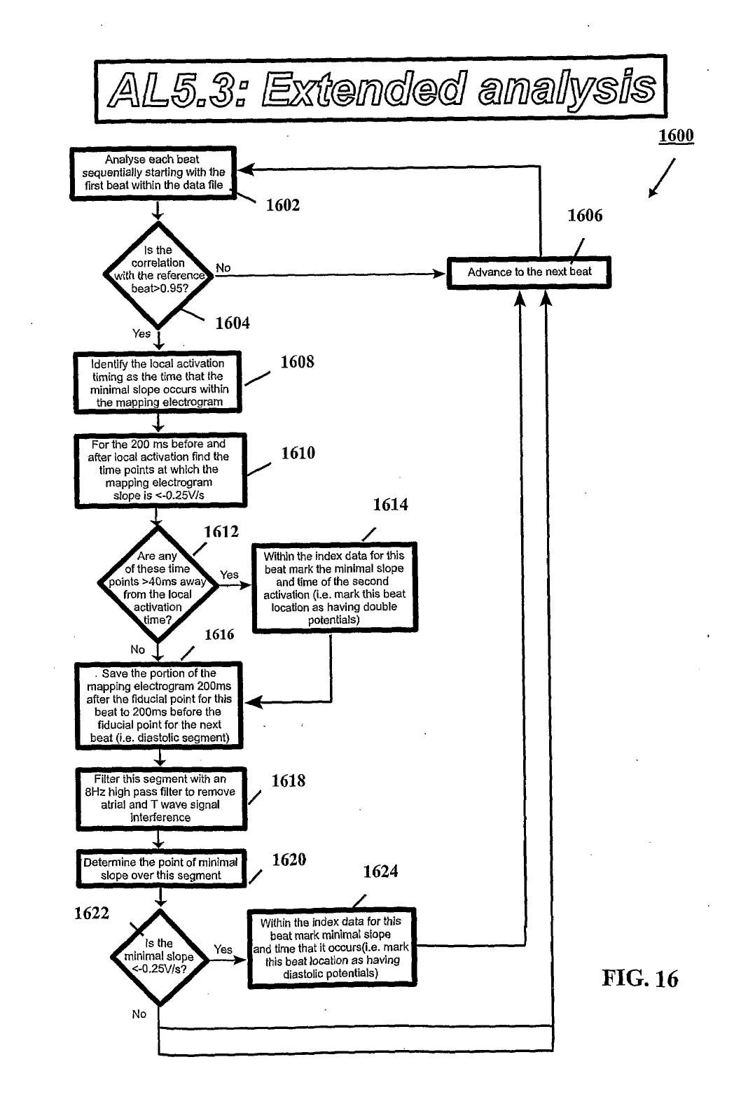

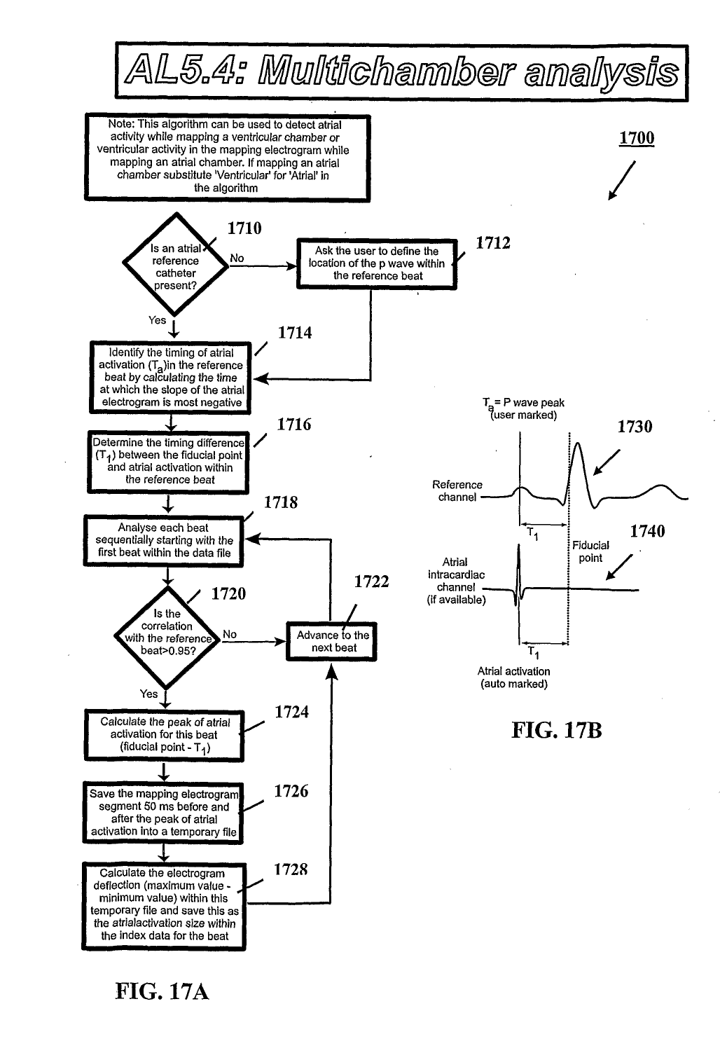

FIG. 13 is a flow diagram providing an overview of an electrogram analysis process 1300, which commences in step 1310. In decision step 1310, a check is made to determine if the process is currently in a real-time mode by determining if electrogram data is currently being recorded. If step 1310 returns true (Yes), processing continues at step 1330. In step 1330, basic substrate mapping is performed only using the sub-processes 1600 and 1700 of FIGS. 16 and 17 to reduce computational requirements. Each of the sub-processes 1600 and 1700 for electrogram fault analysis and main electrogram analysis, respectively, is described in greater detail hereinafter. Processing then terminates. Otherwise, if step 1310 returns false (No), processing continues at step 1312.

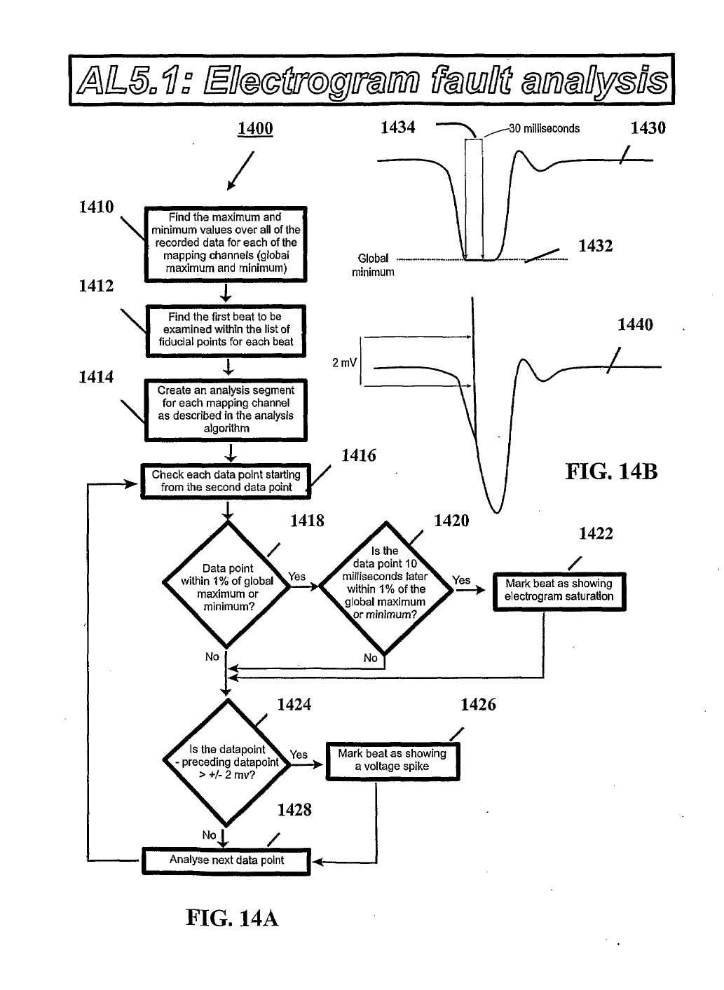

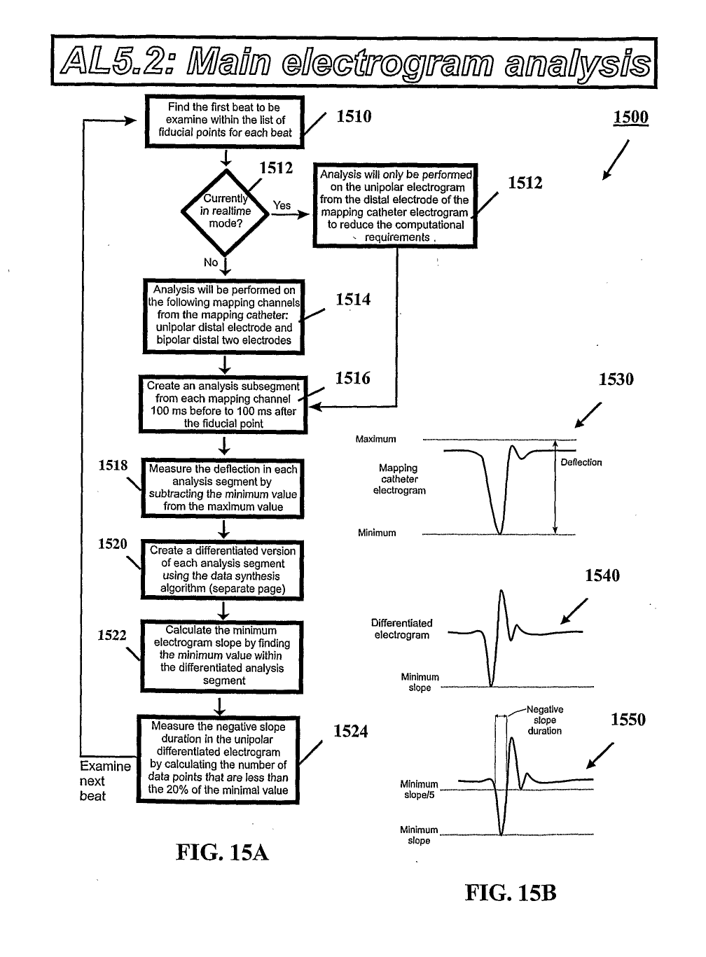

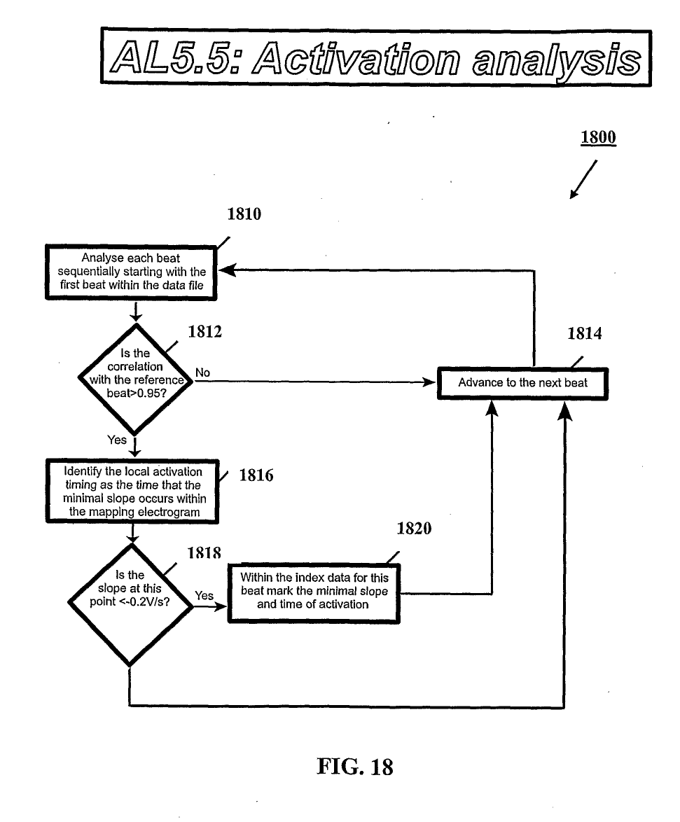

In step 1312, checks for data errors in the mapping channel are performed, using the electrogram fault analysis process 1400 of FIG. 14. In step 1314, basic substrate mapping is performed, using the main electrogram analysis process 1500 of FIG. 15. In step 1316, extended substrate mapping is performed, using the extended electrogram analysis process 1600 of FIG. 16. In step 1318, assessments are made for the presence of electrical activity originating from another chamber (e.g. atrial electrograms), using the multi-chamber analysis process 1700 of FIG. 17. In step 1320, the activation within the cardiac chamber is analysed, using the activation analysis process 1800 of FIG. 18. Processing then terminates.

FIG. 14A is a flow diagram illustrating the electrogram fault analysis process 1400, which commences in step 1410. In step 1410, the maximum and minimum values are found over all of the recorded data for each of the mapping channels (i.e. global maximum and minimum values are found). FIG. 14B shows a global minimum 1432 for recorded data 1430 with a duration 1434 of greater than 30 ms. In step 1412, the first beat to be examined within the list of fiducial points is found for each beat. In step 1414, an analysis segment is created for each mapping channel, as described in the analysis process 1500 of FIG. 15 In step 1416, the program is instructed to perform steps 1418 to 1428 sequentially on every data point within the analysis segment starting from the second data point.

In decision step 1418, a check is made to determine if the current data point is within one percent (1%) of the global maximum or minimum. Again, FIG. 14B shows such an electrogram 1430. If step 1418 returns false (No), processing continues at step 1424. If step 1418 returns true (Yes), processing continues at step 1420. In decision step 1420, a check is made to determine if the data point that is 10 ms later is within one percent (1%) of the global maximum or minimum. If step 1420 returns false (No), processing continues at step 1424. Otherwise, if step 1420 returns true (Yes), processing continues at step 1422. In step 1422, the beat is marked as showing electrogram saturation. Processing then continues at step 1424.

In decision step 1424, a check is made to determine if the data point preceding the current data point differs from the current data point by more than 2 mV. FIG. 14B also shows an electrogram 1440 with a voltage change of 2 mV. If step 1424 returns false (No), processing continues at step 1428. Otherwise, if step 1424 returns true (Yes), processing continues at step 1426. In step 1426, the beat is marked as showing a voltage spike. Processing then continues at step 1428. In step 1428, the next data point is analysed. Processing then returns to step 1416.

FIG. 15A illustrates the main electrogram analysis process 1500 of FIG. 13. Processing commences in step 1510. In step 1510, the first beat to be examined is found within the list of fiducial points for each point. In decision step 1512, a check is made to determine if the process is currently in the real-time mode of operation. If step 1512 returns true (Yes), processing continues at step 1512. In step 1512, analysis is only performed on the unipolar electrogram from the distal electrode of the mapping catheter electrogram to reduce the computational requirements. Processing then continues at step 1516. Otherwise, if step 1512 returns false (No), processing continues at step 1514. In step 1514, the analysis is performed on the following mapping channels from the mapping catheter: unipolar distal electrode and the two bipolar distal electrodes. Processing then continues at step 1516. In step 1516, an analysis subsegement is created from each mapping channel 100 ms before the fiducial point to 100 ms after that fiducial point.

In step 1518, the deflection in each analysis segment is measured by subtracting the minimum value from the maximum value. FIG. 15B illustrates the deflection between the maximum and minimum values of the mapping catheter electrogram 1530. In step 1520, a differentiated version of each analysis segment is created, using the data synthesis process 900 of FIG. 9A. A differentiated electrogram 1540 is depicted in FIG. 15B. In step 1522, the minimal electrogram slope is calculated by finding the lowest value in the differentiated analysis segment. The minimum slope is also depicted in FIG. 15B. In step 1524, the negative slope duration in the unipolar differentiated electrogram is measured by calculating the number of data points that are less than 20% of the minimal value. This is depicted as minimum slope/5 for the differentiated electrogram 1550 with the negative slope duration marked. From step 1524, the next beat is examined and processing continues at step 1510.

FIG. 16 depicts the extended analysis process 1600. Processing commences in step 1602. In step 1602, each beat is analysed sequentially starting with the first beat within the data file. In decision step 1604, a check is made to determine if the correlation with the reference beat is greater than 0.95 (the correlation scale extends from −1.0 to 1.0 with 1.0 representing a perfect match and therefore electrograms with a correlation of 0.95 are highly correlated). If step 1604 returns false (No), processing continues at step 1606. In step 1606, processing advances to the next beat. Processing then continues at step 1602. Otherwise, if step 1604 returns true (Yes), processing continues at step 1608. In the above embodiment, only electrograms that have a high correlation (>0.95) with the reference electrogram are analysed to reduced computational requirements. However, if the program is operating on sufficiently powerful hardware, all electrograms may be analysed allowing the user to decide which electrogram data to incorporate into the substrate map.

In step 1608, the local activation timing is identified as the time that the minimal slope occurs within the mapping electrogram. In step 1610, time points at which the mapping electrogram slope is less than −0.25V/s is found for the 200 ms before and after the local activation. In decision step 1612, a check is made to determine if there are any of these time points greater than 40 ms away from the local activation time. If step 1612 returns false (No), processing continues at step 1616. Otherwise, if step 1612 returns true (Yes), processing continues at step 1614. In step 1614, within the index data for the current beat, the minimal slope and time of second activation are marked (i.e., mark this beat location as having double potentials). Processing then continues at step 1616.

In step 1616, the portion of the mapping electrogram 200 ms after the fiducial point for this beat to 200 ms before the fiducial point for the next beat (i.e. diastolic segment) are saved. In step 1618, this segment is high-pass filtered to remove atrial and T-wave signal interference. The filter may be an 8 Hz high pass filter. In step 1620, the point of minimal slope over the segment is determined. In decision step 1622, a check is made to determine if the minimal slope is less than −0.25 V/s. If step 1622 returns false (No), processing continues at step 1606. Otherwise, if step 1622 returns true (Yes), processing continues at step 1624. In step 1624, within the index data for this beat, the minimal slope and the time that the slope occurs are marked (i.e., this beat location is marked as having diastolic potentials). Processing then continues at step 1606.

FIG. 17A illustrates the multi-chamber analysis process 1700. This algorithm can be used to detect atrial activity while mapping a ventricular chamber or ventricular activity in the mapping electrogram while mapping an atrial chamber. If mapping an atrial chamber, “Ventricular” may be substituted for “Atrial” in the algorithm. Processing commences in step 1710. In decision step 1710, a check is made to determine if an atrial reference catheter is present. If step 1710 returns false (No), processing continues in step 1712. In step 1712, the user is asked to define the location of the P wave within the reference beat. Processing continues at step 1714. Otherwise, if step 1710 returns true (Yes), processing continues at step 1714. In step 1714, the timing of the activation Tₐ in the reference beat by calculating the time at which the slope of the atrial electrogram is most negative. FIG. 17B depicts the reference channel 1730 with timing of the activation Tₐ (p wave peak marked by user). The atrial intracardiac channel (if available) 1740 is also shown with the atrial activation (auto marked). In step 1716, the timing difference T₁ between the fiducial point and atrial activation within the reference beat is determined. In step 1718, each beat is analysed sequentially starting with the first beat within the data file.

In decision step 1720, a check is made to determine if the correlation with the reference beat is greater than 95%. If step 1720 returns false (No), processing continues at step 1722. In step 1722, processing advances to the next beat. Processing then continues at step 1718. Otherwise, if step 1720 returns true (Yes), processing continues at step 1724. In step 1724, the peak of atrial activation is calculated for this beat (fiducial point—T₁). In step 1726, the mapping electrogram segment 50 ms before and after the peak of atrial activation into a temporary file. In step 1728, the electrogram deflection is calculated (maximum value—minimum value) within this temporary file, and this deflection is saved as the atrial activation size within the index data for the beat. Processing then continues at step 1722.

FIG. 18 illustrates the activation analysis process 1320 of FIG. 13. Processing commences in step 1810. In step 1810, each beat is analysed sequentially starting with the first beat within the data file. In decision step 1812, a check is made to determine if the correlation with the reference beat exceeds 95% (0.95). If step 1812 returns false (No), processing continues at step 1814. In step 1814, processing advances to the next beat. Processing then returns to step 1810. If step 1812 returns true (Yes), processing continues at step 1816. In step 1816, the local activation timing is identified as the time that the minimal slope occurs within the mapping electrogram. In decision step 1818, a check is made to determine if the slope at this point is less than 0.2 V/s. If step 1818 returns false (No), processing continues at step 1814. Otherwise, if step 1818 returns true (Yes), processing continues at step 1820. In step 1820, within the index data for the current beat, the minimal slope and activation time are marked. Processing then continues at step 1814.

The automated electrogram analytic steps that have been detailed above describe relatively simple procedures designed to minimise the use of computing resources. Further automated analysis that can be performed includes:

1. Correlation of each recorded intracardiac electrogram with normal and scarred intracardiac electrograms. Typically the user moves the mapping catheter to deep within the scarred region of the myocardium and then records a beat that acts as the (“reference scar beat”). Another beat can then be recorded from normal myocardium (“reference normal beat”). The analysis program compares each recorded intracardiac electrogram with both the reference scar beat and the reference normal beat using Pearson's correlation. These correlations values are recorded within the analysis data for each beat allowing the correlation values and the analysis data to be used to create a scar map (eg. areas from which the recorded intracardiac electrogram showing a correlation of >0.95 may be marked as scar on the map).
2. Analysis with a neural network that had been trained using a database of normal and scarred myocardial beats.

### Display (AL6)

The automated map is a 3-dimensional representation of the cardiac chamber. The 3-dimensional location of each recorded beat is used as the basis of this map. A convex hull algorithm is applied to the locations of all of the beats within the data set. This creates a simple faceted model from the outer (extrema) points ignoring the inner points. A b-spline algorithm may be used to smooth this geometry and create multiple (e.g. 2000) evenly spaced vertices. A convex hull algorithm is then used to join these vertices to create 3-dimensional triangles or faces. The final model of the heart is the collection of faces displayed as a 3-dimensional model. Computer graphics cards are often optimised to display data in this fashion, allowing the model to be rotated, resized and moved interactively by the user in real-time. There are various software development tools that can be used to create software to visualise 3-dimensional models (eg. ‘Matlab’ Mathworks Inc.). These tools may also be used to create controls to enable the user to interact (rotate, zoom and select) with these models.

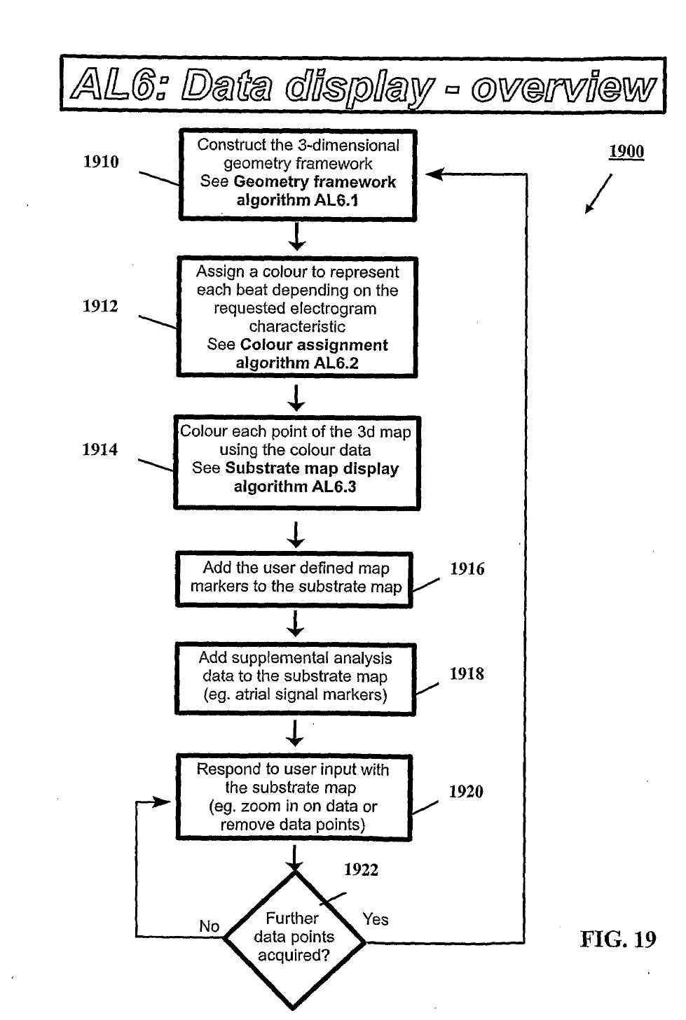

The substrate map is completed by colouring the 3-dimensional model to show the locations of abnormal myocardium. Those parts of the geometry within a specified distance (eg. 5 mm) from the recording site of an electrogram are coloured according to the analysis results for that electrogram. Although the locations of all of the beats are used to create the geometry, only the data from valid beats (e.g. sinus rhythm beats with no data artefacts) is used to colour the final substrate map. Regions of the chamber that have not been mapped (i.e. no electrograms have been recorded from or near that point) may be displayed as light grey. Regions of the chamber that have been mapped may be displayed with a colour depending on the value of electrogram analysis from that region. The minimum negative slope in the distal unipolar electrogram may be the criteria used to colour the substrate map and identify abnormal (i.e. scarred) myocardium. A threshold value may be used to represent colour in only those regions that were abnormal to make the substrate map easier to interpret. Regions that have been mapped but found to be normal (i.e. the electrogram characteristic did not meet the threshold criteria) may be coloured dark grey. Conversely, regions that have been mapped and found to be abnormal may be shown as a distinctive colour such as red to clearly identify abnormal (eg. scarred myocardium) on the substrate map. FIGS. 19-22 illustrate the display process 2100 in detail.

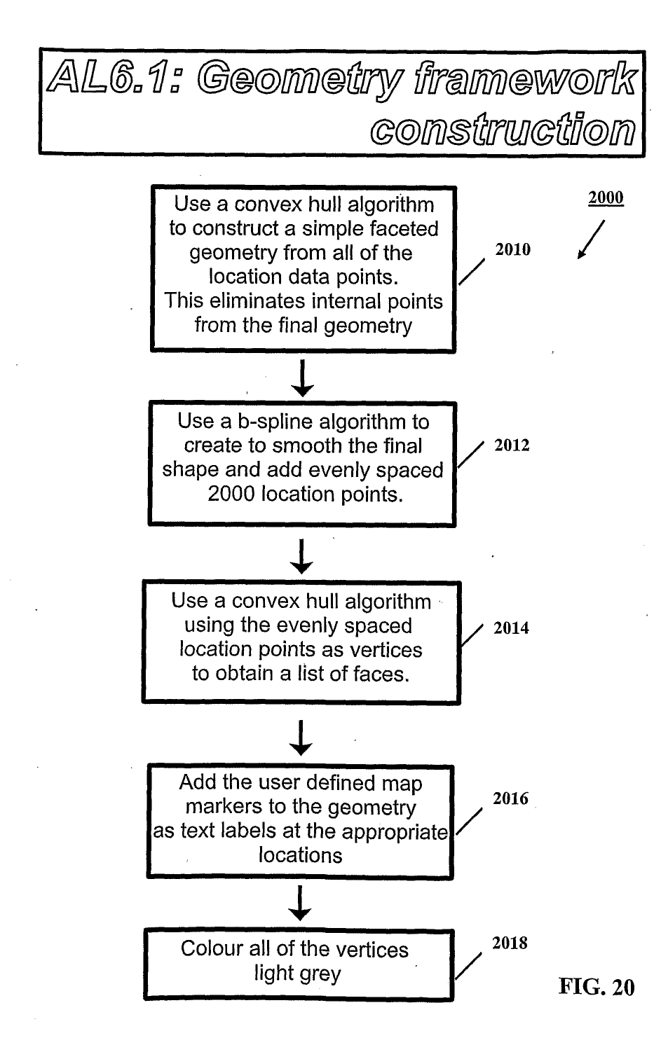

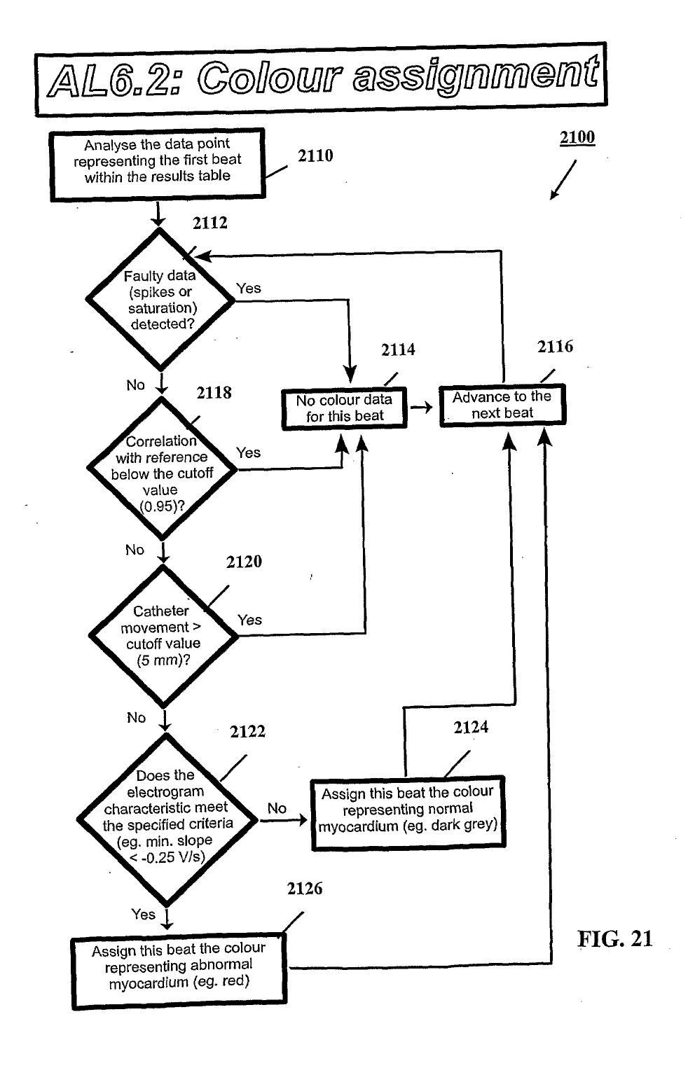

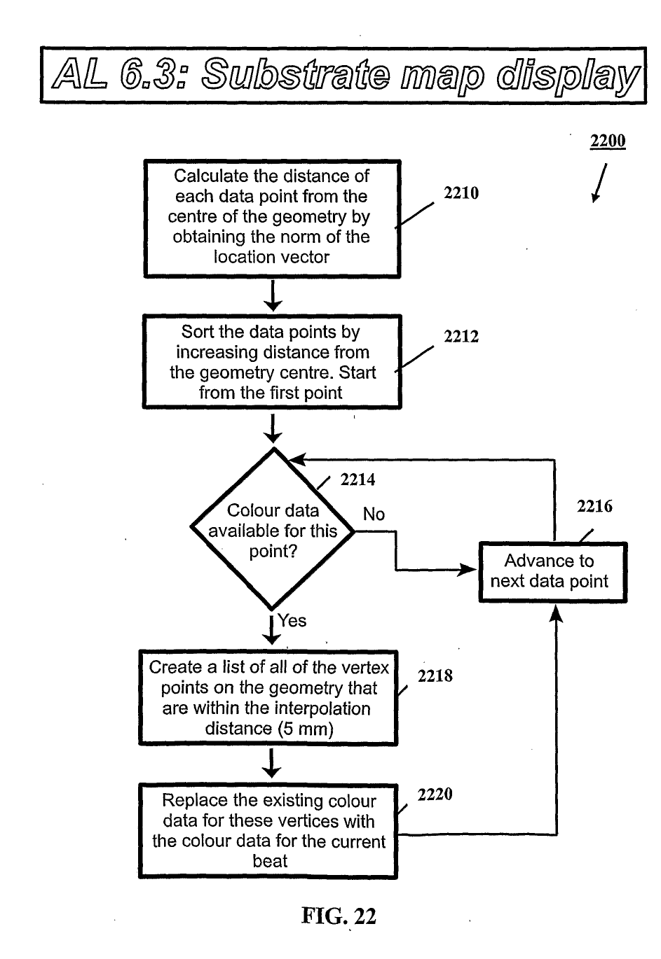

FIG. 19 provides an overview of the data display process 1900, commencing in step 1910. In step 1910, a 3-dimensional geometry framework is constructed. The details of this sub-process 2000 are set forth in FIG. 20. In step 1912, a colour is assigned to represent each beat depending on the requested electrogram characteristic. The details of this sub-process 2100 are set forth in FIG. 21. In step 1914, each point of the 3-d map is coloured using the colour data. The details of this sub-process 2200 are set forth in FIG. 22. In step 1916, the user-defined map markers are added to the substrate map. In step 1918, supplemental analysis data is added to the substrate map (e.g., atrial signal markers). In step 1920, response actions are performed with the substrate map based on user inputs (e.g., zoom in on data or remove data points). In decision step 1922, a check is made to determine if further data points have been acquired. If step 1922 returns false (No), processing returns to step 1920. Otherwise, if step 1922 returns true (Yes), processing continues at step 1910.

FIG. 20 illustrates the process 2000 of geometry framework construction of step 1910 of FIG. 19. Processing commences in step 2010. In step 2010, a simple faceted geometry is constructed from all of the location data points, using a convex hull algorithm. This eliminates internal points from the final geometry. In step 2012, the geometry is smoothed to create the final shape and 2000 evenly spaced location points are added, using a b-spline algorithm. In step 2014, the evenly spaced location points are used as vertices to obtain a list of faces, using the convex hull algorithm. In step 2016, the user defined map markers are added to the geometry as text labels at the appropriate locations. In step 2018, the vertices are coloured, e.g. light grey. Processing of this task then terminates.

FIG. 21 illustrates the colour assignment process 2100 of step 1912 of FIG. 19. Processing commences in step 2110. In step 2110, the data point representing the first beat within the results table is obtained for analysis by steps 2112 to 2126. In decision step 2112, a check is made to detect if the data point involves faulty data (e.g., spikes or saturation). If step 2112 returns false (No), processing continues at step 2118. Otherwise, if step 2112 returns true (Yes), processing continues at step 2114. In step 2114, colour data for the current beat is not assigned. Processing then continues at step 2116. In step 2116, processing advances to the next beat. Processing then returns to step 2112. In decision step 2118, a check is made to determine if the correlation with the reference beat is below the cut-off value (<0.95). If step 2118 returns true (Yes), processing continues at step 2114. Otherwise, if step 2118 returns false (No), processing continues in step 2120.

In decision step 2120, a check is made to determine if the catheter movement is greater than the cut-off value (e.g., a threshold of 5 mm). If step 2120 returns true (Yes), processing continues at step 2114. Otherwise, if step 2120 returns false (No), processing continues at step 2122. In decision step 2122, a check is made to determine if the electrogram characteristic meets specified criteria (e.g., the minimum slope is less than −0.25 V/s). If step 2122 returns false (No), processing continues at step 2124. In step 2124, the current beat is assigned a colour representing normal myocardium tissue (e.g., dark grey). Processing then continues at step 2126. Otherwise, if step 2122 returns false (No), processing continues at step 2126. In step 2126, the current beat is assigned a colour representing abnormal myocardium tissue (e.g., red). Processing then continues at step 2116.

FIG. 22 illustrates the substrate map display process 2200 of step 1914 of FIG. 19. Processing commences in step 2210. In step 2210, the distance of each data point from the centre of the geometry is calculated by obtaining the norm of the location vector. In step 2212, the data points are sorted by their distance from the geometry centre, starting from the closest point and ending with the point that is furthermost from the centre of the geometry. In decision step 2214, a check is made to determine colour data is available for the current data point. If step 2214 returns false (No), processing continues at step 2216. In step 2216, processing advances to the next data point. Processing returns to step 2214. Otherwise, if step 2214 returns true (Yes), processing continues at step 2218. In step 2218, a list of the vertex points on the geometry that are within the interpolation distance (e.g., 5 mm) is created. In step 2220, the existing colour data for these vertices are replaced with the colour data for the current beat. Processing then returns to step 2216.

In the examples given within the preceding sections, a single analysis parameter (eg. electrogram minimum slope) has been used to colour the substrate map. However, the operator may choose to combine two or more analysis parameters to colour the map by combining the two parameters in a mathematical formula (eg. colour data=electrogram minimum slope divided by the electrogram duration slope). Alternatively, a neural network that had been trained on sample data sets may be used to evaluate the analysis results and provide the colour data. Expert system technology may also be utilised in another embodiment of the invention to combine the results of multiple analysis results into a single colour value for each beat.

In the above-described embodiment of the invention, a data threshold (in this case −0.25V/s) has been used to colour the map in a binary fashion (i.e. only two values with red=scar and dark grey=normal) for ease of interpretation. In other embodiments, a smooth range of colours (e.g. 256 colours ranging from red to blue) may be used to represent a gradation of data values.

### Substrate Map Review (AL7)

By selecting a region of the 3-dimensional substrate map, the operator can obtain a detailed analysis of that location. Depending on software inputs that the operator has provided (e.g. by activating software dialog buttons) before selecting the region, one or many of the following actions are performed by the mapping system in response to the user input:

(1) Find the electrogram data recording site closest to the region of interest (i.e. the point selected by the user) and then display the mapping catheter electrograms that have been recorded from that site; and

(2) Display the mapping catheter electrograms that have been recorded near the region of interest. These electrograms are scaled and displayed in the zoomed electrogram window with the correct orientation to each other. This allows the user to check the scar border zones and observe the changes in voltage and slope. The user is also able to check if any artefacts are present in the mapping electrogram that have not been detected by the automatic faulty electrogram checking process 1400 of FIG. 14.

The operator can use the zoomed-electrogram window to exclude beats that have incorrect data (due to an ectopic beat or an artefact in the electrogram trace) from the substrate map.

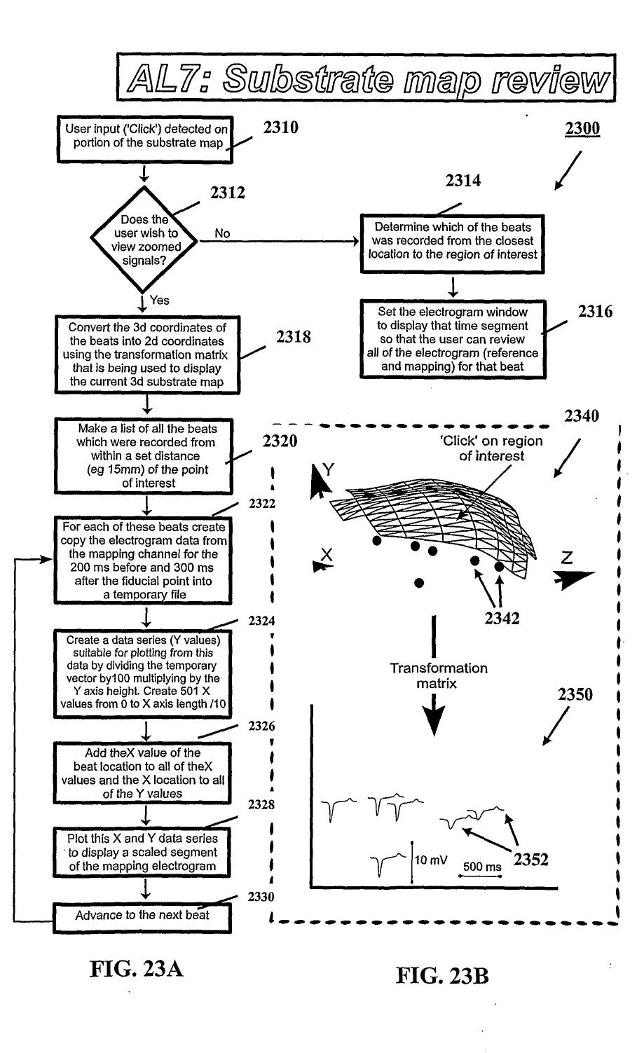

FIG. 23A illustrates the process of substrate map review 2300. Processing commences in step 2310. In step 2310, operator input (“click”) is detected on the portion of the substrate map. In decision step 2312, a check is made to determine if the operator wants to view zoomed electrogram signals (i.e. view a 2 dimensional map showing the electrograms from a distinct portion of the heart chamber centred around the selected point). Typically, the operator activates a software switch to indicate how the program should respond to a ‘mouse click’ over the 3d substrate map. If step 2312 returns false (No), processing continues at step 2314. In step 2314, the beat or beats are determined that have been recorded as being from the closest location to the region of interest, based on the operator input of step 2310. In step 2316, the electrogram window to display that time segment is set, so that the operator can review all of the electrogram (reference and mapping electrograms) for that beat. Processing of this event then terminates. Otherwise, if step 2312 returns true (Yes), processing continues at step 2318.

In step 2318, the 3-dimensional coordinates (X, Y, & Z) of all of the recorded beats are converted into 2-dimensional coordinates using an orthographic transformation matrix. A transformation matrix is a four-by-four matrix that can be used to position a 3-dimensional point in the correct position for any given viewpoint. Therefore, if all of the electrogram positions are multiplied by the transformation matrix that is currently being applied to view the 3-dimensional substrate plot, the electrograms is shown with the exact same orientation as the substrate map. This allows the user to compare the colours visible on the 3-dimensional map with the actual electrograms used to calculate the colour data.

The transformation matrix allows 3-dimensional objects to be viewed on a 2-dimensional display device (such as a computer monitor) from a variety of different viewpoints. After a 3-dimensional point has been multiplied by the transformation matrix for a specific viewpoint, new X, Y and Z values are obtained. The new ‘X’ value represents left and right on the computer monitor, ‘Y’ represents up and down on the computer monitor. The ‘Z’ value can be ignored because this value represents distance away from and towards the monitor, which can not be shown using a 2-dimensionla display device.

Using the transformation matrix, a 2-dimensional map can be created showing the electrograms that were recorded from a specific location within the cardiac chamber. The user can specify the size of the area from which to collect electrograms. If the user specifies a distance of 15 mm for example, only those electrograms that are recorded within 15 mm of the central point are displayed.

After the program has determined which of the multitude of electrograms to display, an X and Y data series is created for each electrogram, allowing the electrograms to be correctly scaled and positioned on the 2-dimensional map. The first data value is position at the location from which each electrogram was recorded using the X and Y values corrected for the current viewpoint. The rest of the electrogram is then drawn to the right of this initial point. The displayed electrogram is orientated so that a positive voltage value within the electrogram data file results in an upward deflection of the electrogram depiction on the 2-dimensional plot. The electrograms may be scaled so that the distance between 0 and 10 mV is 10% of the total 2-dimensional plot Y axis. The total 501 ms data segment is scaled so that the segment occupies 10% of the X axis.

Steps 2322 to 2330 describe in detail how the appropriate X and Y series is created for each electrogram so that the series are all positioned correctly and drawn to the correct scale. In step 2322, for each of the beats, a copy of the electrogram data is created from the mapping channel for the 200 ms before and 300 ms after the fiducial point into a temporary series. In step 2324, the Y series is created by dividing each value in the temporary series by 100 and multiplying by the length of the Y axis. For example, if the user is viewing all electrograms within 15 mm of the central point, the Y axis is 30 mm long (−15 mV to +15 mV), so each electrogram voltage value is divided by 100 and multiplied by 30 (i.e., 10 mV=3 mm which is 10% of the Y axis length).

The corresponding X series comprises 501 evenly spaced values from 0.0 to 10% of the X. axis length. In step 2326, the viewpoint-adjusted, X and Y values of the point from which each beat has been recorded is added to this X series and Y series, respectively. For example, if an electrogram had been recorded from a corrected location of X=3.4 mm, Y=5.6 mm and the 2-dimensional map is set to show all electrograms within 15 mm of the central point, this electrogram's X series comprises 501 evenly-spaced values from 3.4 to 3.7. The Y series comprises 501 values that vary depending on the voltage at that particular time point (eg. if the voltage was +10 mV, the corresponding Y value is 5.6+0.3=5.9).

In step 2330, processing advances to the next beat. Processing then returns to step 2322.

In FIG. 23B, a portion of the 3-dimensional substrate map 2340 and the associated 2-dimensional zoomed electrogram map 2350 are shown. The circular markers represent the 3-dimensional locations, from which these six electrograms were recorded. The electrograms from the right side of this region of interest 2342 were recorded from a region of scarred myocardium. The corresponding electrograms 2352 can seen to have a lower voltage (i.e. are smaller) and do not have a sharp downward deflection (i.e. the electrograms have a low slope).

A variety of alternative criteria other than spatial location alone may also be used to arrange the electrograms on the 2-dimensional zoomed electrogram window. Using this technique specialised plots can be created that allow aberrant beats to be easily identified. The ‘X’ and ‘Y’ location that each beat is drawn at depends on the result of two different analysis results for that beat. For example, the time of atrial activation is shown on the ‘X’ axis and the voltage in the ventricular reference catheter is shown in the ‘Y’ axis. The resulting plot shows a central region, which has many overlapping electrograms (normal zone) and a peripheral region with relatively few electrograms. By this means, the user can quickly identify aberrant beats that may have escaped detection by the beat classification algorithm as these beats generally have abnormal values for these two variables. These aberrant beats are hence easily visible at the corners of the resulting plot.

The zoomed electrogram window is a dynamic displaying allowing the user to interact with and modify the electrogram data set. When the user clicks on the zoomed electrogram window, the program records the ‘X’ and ‘Y’ locations of the user click. These ‘X’ and ‘Y’ values are converted into the appropriate data values (eg. in the preceding example the ‘X’ axis is used to represent the time of atrial activation). By this means, the beat that the user selected on the zoomed electrogram window can be identified within the data set. Depending on the software toggles that user has selected a variety of actions can be performed on this beat (eg. identifying this beat as showing as particular feature, removing data from this beat from the substrate map, or ‘jumping’ to this time point within the electrogram window so that this beat can be examined in greater detail).

### Substrate Map Enhancement

After assessing which areas have not been studied during the initial rapid mapping phase, the operator can then move the catheter to these areas of interest. If these areas have not been adequately mapped, the new electrogram beats are recorded from sites more extreme than the existing data. One advantage of the map construction method described hereinbefore is that the old data points do not need to be deleted, because those data points do not contribute to the colour of the map once the geometry has been expanded (i.e. the data points are too far from the surface of the map to contribute to the colour of the automated map).

Alternatively, if the map appears to be satisfactory, the operator can proceed with the case. During the rest of the case, the system may continue to record data. As only a few data channels (reference channels and mapping) and the location data need to be recorded, this data may easily be stored and analysed by most modern computing platforms. This ongoing data acquisition adds extra detail to the substrate map with incidental catheter movement without any extra time burden for the operators.

### Computer Implementation

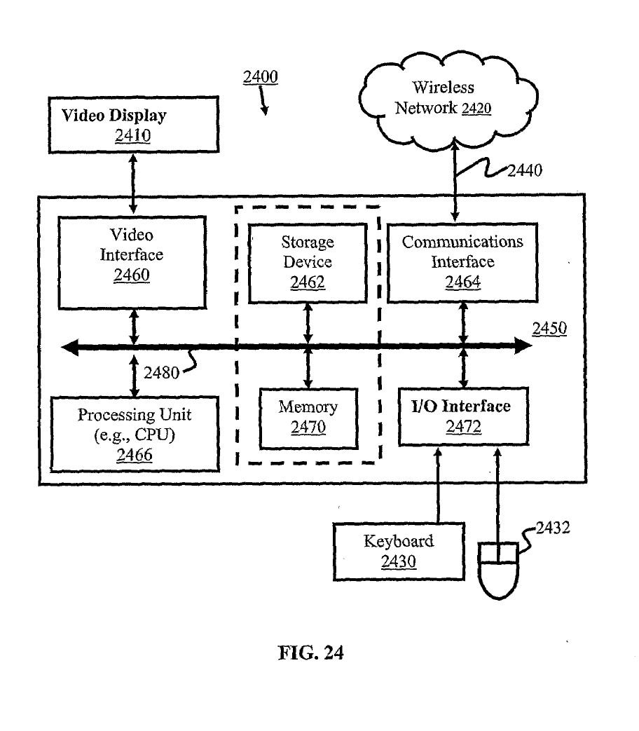

The methods of automated processing of electrophysiological data may be practiced using one or more general-purpose computer systems and handheld devices, in which the processes of FIGS. 1 to 23 may be implemented as software, such as an application program executing within the computer system or handheld device. In particular, the steps of the method of automated mapping and indexing of electrophysiological data are effected, at least in part, by instructions in the software that are carried out by the computer. Software may include one or more computer programs, including application programs, an operating system, procedures and rules. The instructions may be formed as one or more code modules, each for performing one or more particular tasks. The software may be stored in a computer readable medium, comprising one or more of the storage devices described below, for example. The software is loaded into the computer from the computer readable medium and then executed by the computer. A computer readable medium having such software recorded on it is a computer program product. An example of a computer system 2400 with which the embodiments of the invention may be practiced is depicted in FIG. 24.

In particular, the software may be stored in a computer readable medium, comprising one or more of the storage devices described hereinafter. The software is loaded into the computer from the computer readable medium and then carried out by the computer. A computer program product comprises a computer readable medium having such software or a computer program recorded on the medium that can be carried out by a computer. The use of the computer program product in the computer may effect an advantageous apparatus for ensuring data quality and integrity of a data set derived from a data source in accordance with the embodiments of the invention.

The computer system 2400 may comprise a computer 2450, a video display 2410, and one or more input devices 2430, 2432. For example, an operator can use a keyboard 2430 and/or a pointing device such as the mouse 2432 (or touchpad, for example) to provide input to the computer. The computer system may have any of a number of other output devices comprising line printers, laser printers, plotters, and other reproduction devices connected to the computer. The computer system 2400 can be connected to one or more other computers via a communication interface 2464 using an appropriate communication channel 2440 such as a modem communications path, a computer network, a wireless LAN, or the like. The computer network may comprise a local area network (LAN), a wide area network (WAN), an Intranet, and/or the Internet 2420, for example.

The computer 2450 may comprise one or more central processing unit(s) 2466 (simply referred to as a processor hereinafter), memory 2470 which may comprise random access memory (RAM) and read-only memory (ROM), input/output (IO) interfaces 2472, a video interface 2460, and one or more storage devices 2462. The storage device(s) 2462 may comprise one or more of the following: a floppy disc, a hard disc drive, a magneto-optical disc drive, CD-ROM, DVD, a data card or memory stick, magnetic tape or any other of a number of non-volatile storage devices well known to those skilled in the art. For the purposes of this description, a storage unit may comprise one or more of the memory 2470 and the storage devices 2462. The storage devices 2462 may incorporate data compression technology to increase the recording capacity of the system.

Each of the components of the computer 2450 is typically connected to one or more of the other devices via one or more buses 2480, depicted generally in FIG. 24, that in turn comprise data, address, and control buses. While a single bus 2480 is depicted in FIG. 24, it will be well understood by those skilled in the art that a computer or other electronic computing device such as a PDA or cellular phone may have several buses including one or more of a processor bus, a memory bus, a graphics card bus, and a peripheral bus. Suitable bridges may be utilised to interface communications between such buses. While a system using a processor has been described, it will be appreciated by those skilled in the art that other processing units capable of processing data and carrying out operations may be used instead without departing from the scope and spirit of the invention.

The computer system 2400 is simply provided for illustrative purposes and other configurations can be employed without departing from the scope and spirit of the invention. Computers with which the embodiment can be practiced comprise IBM-PC/ATs or compatibles, one of the Macintosh™ family of PCs, Sun Sparcstation™, a workstation or the like. The foregoing are merely examples of the types of computers with which the embodiments of the invention may be practiced. Typically, the processes of the embodiments, described hereinafter, are resident as software or a program recorded on a hard disk drive as the computer readable medium, and read and controlled using the processor. Intermediate storage of the program and intermediate data and any data fetched from the network may be accomplished using the semiconductor memory.

In some instances, the program may be supplied encoded on a CD-ROM or a floppy disk, or alternatively could be read from a network via a modem device connected to the computer, for example. Still further, the software can also be loaded into the computer system from other computer readable medium comprising magnetic tape, a ROM or integrated circuit, a magneto-optical disk, a radio or infra-red transmission channel between the computer and another device, a computer readable card such as a PCMCIA card, and the Internet and Intranets comprising email transmissions and information recorded on websites and the like. The foregoing is merely an example of relevant computer readable mediums. Other computer readable mediums may be practiced without departing from the scope and spirit of the invention.

### CONCLUSION

The embodiments of the invention enable recording long sections or the entirety of a cardiac electrophysiological study and automatically identifying the location of every beat within this data set. These beats may then be analysed for numerous features and adding the results of this analysis into an index table for each beat. A table of indexed criteria may be used to select and review beats that meet specific user defined criteria. The spatial location from which the beat was recorded may be added into the index of information for that beat. The automated analysis data and spatial information may be compiled within the indexed data. This may be done to create spatial maps representing abnormal locations within the myocardium (automated substrate maps) and/or the spread of activation for a specific cardiac rhythm (automated activation maps).

The user can define a reference beat, which may then be used to select other similar beats within the data set for analysis. An averaged differentiated unipolar electrogram may be used to determine the location of myocardial scarring and/or the time of activation at various locations throughout the heart.

The method can display electrograms in a 3-dimensional map that can be altered interactively. The user can change the cutoff values for correlation with the reference electrogram or the threshold value that is used to represent abnormal tissue. A convex hull algorithm may be used to choose data points located at the periphery (i.e. data points that are likely to be in good contact with the myocardial wall) for display on the substrate map.

The electrogram data may be displayed associated with a 3-dimensional point on the substrate map when the user selects that point.

The system can process electrogram data in real time, enabling a highly accurate map to be formed using electrogram recording that are acquired serendipitously, i.e. the user may be attempting to locate the catheter at a particular site but it falls into another area that required analysis.

A small number of embodiments of the invention regarding methods, apparatuses, and computer program products for automated processing of electrophysiological data have been described. In the light of the foregoing, it will be apparent to those skilled in the art in the light of this disclosure that various modifications and/or substitutions may be made without departing from the scope and spirit of the invention.

1. A method of automated processing of intracardiac electrophysiological data, said method comprising the steps of:
recording electrogram data and corresponding spatial location data of an electrode recording said electrogram data, said recorded electrogram data comprising a plurality of beats;
defining at least one reference channel containing a reference beat against which beats of said recorded electrogram data are compared;
examining said recorded electrogram data and defining a temporal location for each beat of said recorded electrogram data;
creating an index of said temporal locations and other information of said beats within said recorded electrogram data;
analysing in real-time at least one electrophysiological feature of said recorded electrogram data suggestive of a physiological condition; and
providing an updated index wherein said other information comprises results of said analysis.

2. The method according to claim 1, wherein said physiological condition comprises a local abnormality.

3. The method according to claim 2, wherein said local abnormality comprises myocardial scarring.

4. The method according to claim 1, further comprising the step of generating a scatter plot graphically representing information based on said recorded electrogram data.

5. The method according to claim 1, further comprising the step of generating dependent upon said updated index a multidimensional substrate map comprising a plurality of vertices each having a corresponding spatial location and graphically representing information based on said recorded electrogram data.

6. The method according to claim 5, wherein at least a portion of said graphically represented information represents scarred myocardial tissue.

7. The method according to claim 5, wherein at least a portion of said graphically represented information represents healthy myocardial tissue.

8. The method according to claim 5, wherein said information is graphically represented using colour.

9. The method according to claim 5, wherein said multidimensional substrate map is a 3-dimensional substrate map.

10. The method according to claim 5, further comprising the step of excluding from said substrate map data for beats recorded within the cardiac chamber that have insufficient contact with a wall of said cardiac chamber.

11. The method according to claim 10, wherein said excluding step comprises using a convex hull algorithm to select beats recorded at extreme locations for display in said substrate map.

12. The method according to claim 1, further comprising the step of triggering an alarm dependent upon said updated index.

13. The method according to claim 1, further comprising the step of choosing at least one electrogram channel to be recorded, dependent upon the application.

14. The method according to claim 13, wherein said chosen channel comprises at least one of ECG channels, a ventricular reference channel, an atrial reference channel, and a distal mapping catheter.

15. The method according to claim 1, further comprising the step of analysing as a background task at least one electrophysiological feature of said recorded electrogram data.

16. The method according to claim 1, wherein said real-time analysing step comprises checking said recorded electrogram data for at least one feature suggestive of a fault condition or an artefact.

17. The method according to claim 1, wherein said defining step comprises determining fiducial points that define the temporal location of each beat recorded within said reference channel.

18. The method according to claim 17, further comprising the steps of calculating a differentiated reference electrogram and finding points at which said differentiated reference electrogram exceeds a predefined beat threshold to determine said fiducial points.

19. The method according to claim 18, wherein said step of calculating said differentiated electrogram comprises subtracting a data point from said recorded electrogram data at a specified temporal location before the time zero point from a data point from said recorded electrogram data at another specified temporal location after the time zero point.

20. The method according to claim 19, wherein said step of calculating said differentiated electrogram further comprises dividing the subtraction value by the number of data samples between said data points and multiplying the result by a sampling rate of said recorded electrogram data.

21. The method according to claim 17, wherein said real-time analysing step comprises determining an analysis segment of said recorded electrogram data.

22. The method according to claim 21, wherein said analysis segment is created as a sub-segment a predetermined time before and after the fiducial point for each beat in said recorded electrogram data.

23. The method according to claim 21, wherein said real-time analysing step comprises finding a minimum slope once for each analysis segment for each beat.

24. The method according to claim 23, wherein said real-time analysing step comprises detecting when a local activation occurs in said analysis segment in reference to said fiducial point in said beat of said recorded electrogram data.

25. The method according to claim 24, where said detecting step comprises determining a temporal location at which said minimum slope occurs.

26. The method according to claim 1, wherein said real-time analysing step comprises correlating at least one reference beat of said reference channel with beats of said recorded electrogram data.

27. The method according to claim 1, wherein said feature analysed in real time comprises the minimum slope in a differentiated unipolar electrogram in a distal mapping catheter electrode.

28. A computer program product comprising a computer readable medium having recorded therein a computer program for automated processing of intracardiac electrophysiological data, said computer program product comprising:
computer program code means for recording electrogram data and corresponding spatial location data of an electrode recording said electrogram data, said recorded electrogram data comprising a plurality of beats;
computer program code means for defining at least one reference channel containing a reference beat against which beats of said recorded electrogram data are compared; computer program code means for examining said recorded electrogram data and defining a temporal location for each beat of said recorded electrogram data;
computer program code means for creating an index of said temporal locations and other information of said beats within said recorded electrogram data;
computer program code means for analysing in real-time at least one electrophysiological feature of said recorded electrogram data suggestive of a physiological condition; and
computer program code means for providing an updated index wherein said other information comprises results of said analysis.

29. The computer program product according to claim 28, wherein said physiological condition comprises a local abnormality.

30. The computer program product according to claim 29, wherein said local abnormality comprises myocardial scarring.

31. The computer program product according to claim 28, further comprising computer program code means for generating a scatter plot graphically representing information based on said recorded electrogram data.

32. The computer program product according to claim 28, further comprising computer program code means for generating dependent upon said updated index a multidimensional substrate map comprising a plurality of vertices each having a corresponding spatial location and graphically representing information based on said recorded electrogram data.

33. The computer program product according to claim 32, wherein at least a portion of said graphically represented information represents scarred myocardial tissue.

34. The computer program product according to claim 32, wherein at least a portion of said graphically represented information represents healthy myocardial tissue.

35. The computer program product according to claim 32, wherein said information is graphically represented using colour.

36. The computer program product according to claim 32, wherein said multidimensional substrate map is a 3-dimensional substrate map.

37. The computer program product according to claim 32, further comprising computer program code means for excluding from said substrate map data for beats recorded within the cardiac chamber that have insufficient contact with a wall of said cardiac chamber.

38. The computer program product according to claim 37, wherein said computer program code means for excluding comprises computer program code means for using a convex hull algorithm to select beats recorded at extreme locations for display in said substrate map.

39. The computer program product according to claim 28, further comprising computer program code means for triggering an alarm dependent upon said updated index.

40. The computer program product according to claim 28, further comprising computer program code means for choosing at least one electrogram channel to be recorded, dependent upon the application.

41. The computer program product according to claim 40, wherein said chosen channel comprises at least one of ECG channels, a ventricular reference channel, an atrial reference channel, and a distal mapping catheter.

42. The computer program product according to claim 28, further comprising computer program code means for analysing as a background task at least one electrophysiological feature of said recorded electrogram data.

43. The computer program product according to claim 28, wherein said computer program code means for real-time analysing comprises computer program code means for checking said recorded electrogram data for at least one feature suggestive of a fault condition or an artefact.

44. The computer program product according to claim 28, wherein said computer program code means for defining comprises computer program code means for determining fiducial points that define the temporal location of each beat recorded within said reference channel.

45. The computer program product according to claim 44, further comprising computer program code means for calculating a differentiated reference electrogram and finding points at which said differentiated reference electrogram exceeds a predefined beat threshold to determine said fiducial points.

46. The computer program product according to claim 45, wherein said computer program code means for calculating said differentiated electrogram comprises computer program code means for subtracting a data point from said recorded electrogram data at a specified temporal location before the time zero point from a data point from said recorded electrogram data at another specified temporal location after the time zero point.

47. The computer program product according to claim 46, wherein said computer program code means for calculating said differentiated electrogram further comprises computer program code means for dividing the subtraction value by the number of data samples between said data points and multiplying the result by a sampling rate of said recorded electrogram data.

48. The computer program product according to claim 44, wherein said computer program code means for real-time analysing comprises computer program code means for determining an analysis segment of said recorded electrogram data.

49. The computer program product according to claim 48, wherein said analysis segment is created as a sub-segment a predetermined time before and after the fiducial point for each beat in said recorded electrogram data.

50. The computer program product according to claim 48, wherein said computer program code means for real-time analysing comprises computer program code means for finding a minimum slope once for each analysis segment for each beat.

51. The computer program product according to claim 50, wherein said computer program code means for real-time analysing comprises computer program code means for detecting when a local activation occurs in said analysis segment in reference to said fiducial point in said beat of said recorded electrogram data.

52. The computer program product according to claim 51, where said computer program code means for detecting comprises computer program code means for determining a temporal location at which said minimum slope occurs.

53. The computer program product according to claim 28, wherein said computer program code means for real-time analysing step comprises correlating at least one reference beat of said reference channel with beats of said recorded electrogram data.

54. The computer program product according to claim 28, wherein said feature analysed in real time comprises the minimum slope in a differentiated unipolar electrogram in a distal mapping catheter electrode.

55. An apparatus for automated processing of intracardiac electrophysiological data, said apparatus comprising: means for recording electrogram data and corresponding spatial location data of an electrode recording said electrogram data, said recorded electrogram data comprising a plurality of beats; means for defining at least one reference channel containing a reference beat against which beats of said recorded electrogram data are compared; means for examining said recorded electrogram data and defining a temporal location for each beat of said recorded electrogram data; means for creating an index of said temporal locations and other information of said beats within said recorded electrogram data; means for analysing in real-time at least one electrophysiological feature of said recorded electrogram data suggestive of a physiological condition; and means for providing an updated index wherein said other information comprises results of said analysis.

56. The apparatus according to claim 55, wherein said physiological condition comprises a local abnormality.

57. The apparatus according to claim 56, wherein said local abnormality comprises myocardial scarring.

58. The apparatus according to claim 55, further comprising means for generating a scatter plot graphically representing information based on said recorded electrogram data.

59. The apparatus according to claim 55, further comprising means for generating dependent upon said updated index a multidimensional substrate map comprising a plurality of vertices each having a corresponding spatial location and graphically representing information based on said recorded electrogram data.

60. The apparatus according to claim 59, wherein at least a portion of said graphically represented information represents scarred myocardial tissue.

61. The apparatus according to claim 59, wherein at least a portion of said graphically represented information represents healthy myocardial tissue.

62. The apparatus according to claim 59, wherein said information is graphically represented using colour.

63. The apparatus according to claim 69, wherein said multidimensional substrate map is a 3-dimensional substrate map.

64. The apparatus according to claim 59, further comprising means for excluding from said substrate map data for beats recorded within the cardiac chamber that have insufficient contact with a wall of said cardiac chamber.

65. The apparatus according to claim 64, wherein said means for excluding comprises means for using a convex hull algorithm to select beats recorded at extreme locations for display in said substrate map.

66. The apparatus according to claim 55, further comprising means for triggering an alarm dependent upon said updated index.

67. The apparatus according to claim 55, further comprising means for choosing at least one electrogram channel to be recorded, dependent upon the application.

68. The apparatus according to claim 67, wherein said chosen channel comprises at least one of ECG channels, a ventricular reference channel, an atrial reference channel, and a distal mapping catheter.

69. The apparatus according to claim 55, further comprising means for analysing as a background task at least one electrophysiological feature of said recorded electrogram data.

70. The apparatus according to claim 55, wherein said means for real-time analysing comprises means for checking said recorded electrogram data for at least one feature suggestive of a fault condition or an artefact.

71. The apparatus according to claim 55, wherein said means for defining comprises means for determining fiducial points that define the temporal location of each beat recorded within said reference channel.

72. The apparatus according to claim 71, further comprising means for calculating a differentiated reference electrogram and finding points at which said differentiated reference electrogram exceeds a predefined beat threshold to determine said fiducial points.

73. The apparatus according to claim 72, wherein said means for calculating said differentiated electrogram comprises means for subtracting a data point from said recorded electrogram data at a specified temporal location before the time zero point from a data point from said recorded electrogram data at another specified temporal location after the time zero point.

74. The apparatus according to claim 73, wherein said means for calculating said differentiated electrogram further comprises means for dividing the subtraction value by the number of data samples between said data points and multiplying the result by a sampling rate of said recorded electrogram data.

75. The apparatus according to claim 71, wherein said means for real-time analysing comprises means for determining an analysis segment of said recorded electrogram data.

76. The apparatus according to claim 75, wherein said analysis segment is created as a sub-segment a predetermined time before and after the fiducial point for each beat in said recorded electrogram data.

77. The apparatus according to claim 75, wherein said means for real-time analysing comprises means for finding a minimum slope once for each analysis segment for each beat.

78. The apparatus according to claim 77, wherein said means for real-time analysing comprises means for detecting when a local activation occurs in said analysis segment in reference to said fiducial point in said beat of said recorded electrogram data.

79. The apparatus according to claim 78, where said means for detecting comprises means for determining a temporal location at which said minimum slope occurs.

80. The apparatus according to claim 55, wherein said means for realtime analysing step comprises correlating at least one reference beat of said reference channel with beats of said recorded electrogram data.

81. The apparatus according to claim 55, wherein said feature analysed in real time comprises the minimum slope in a differentiated unipolar electrogram in a distal mapping catheter, electrode.

82. A system for automated processing of intracardiac electrophysiological data, said system comprising:
a memory for storing data and a computer program; and
a processor coupled to said memory executing said computer program, said computer program comprising instructions for:
recording electrogram data and corresponding spatial location data of an electrode recording said electrogram data, said recorded electrogram data comprising a plurality of beats;
defining at least one reference channel containing a reference beat against which beats of said recorded electrogram data are compared;
examining said recorded electrogram data and defining a temporal location for each beat of said recorded electrogram data;
creating an index of said temporal locations and other information of said beats within said recorded electrogram data;
analysing in real-time at least one electrophysiological feature of said recorded electrogram data suggestive of a physiological condition; and
providing an updated index wherein said other information comprises results of said analysis.

83. The system according to claim 82, further comprising a monitor for displaying graphical data.

84. The system according to claim 82, wherein said computer program further comprises instructions for generating dependent upon said updated index a multidimensional substrate map comprising a plurality of vertices each having a corresponding spatial location and graphically representing information based on said recorded electrogram data.

85. The system according to claim 82, wherein said computer program further comprises instructions for triggering an alarm dependent upon said updated index.

86. The system according to claim 85, further comprising an alarm mechanism producing an audible, visual, or both alarm signal.
